(19) United States
(12) Patent Application Publication
(10) Pub. No.: US 2021/0095244 A1
(43) Pub. Date: Apr. 1, 2021

(54) Methods and Compositions for Storing Bacteria

(71) Applicant: Emma Allen-Vercoe, Ontario (CA)

(72) Inventor: Kathleen Schroeter, Ontario (CA)

(21) Appl. No.: 16/607,921

(22) PCT Filed: Apr. 27, 2018
(86) PCT No.: PCT/IB2018/000519

§ 371 Date: Oct. 24, 2019

Related U.S. Application Data
(60) Provisional application No. 62/491,739, filed on Apr. 28, 2017.

(51) Int. Cl. C12N 1/20; A61K 35/74

(52) U.S. Cl. CPC C12N 1/20; A61K 35/74; C12N 2500/32; C12N 2500/34; C12N 2500/50

(57) ABSTRACT

Methods and compositions for culturing and preserving bacteria are described herein, wherein the cultured and preserved bacteria exhibit improved viability/growth when compared to bacteria preserved/stored by means other than the subject methods. More particularly, methods and compositions for improving bacterial viability following cryopreservation are disclosed herein.

### RELATED APPLICATIONS

This application claims priority of U.S. Provisional Application No. 62/491,739, filed Apr. 28, 2017, the entirety of which is incorporated herein by reference for all purposes.

### FIELD OF INVENTION

Disclosed herein are methods and compositions for culturing and preserving/storing bacteria, wherein the cultured and preserved/stored bacteria exhibit improved viability/growth when compared to bacteria preserved/stored by means other than the subject methods. More particularly, methods and compositions for improving bacterial viability following cryopreservation are disclosed herein.

### BACKGROUND OF INVENTION

There are many methods for storing bacteria; however, each storage method chosen for each particular bacterium is a function of bacterial compatibility, experimental purpose and cell viability. Typically, the storage period of a bacterium increases as the storage temperature decreases. Once the temperature is below the freezing point, cryoprotectants may be used to reduce cell damage caused by the freezing process.

### BRIEF DESCRIPTION OF THE FIGURES

The present invention will be further explained with reference to the attached drawings, wherein like structures are referred to by like numerals throughout the several views. The drawings shown are not necessarily to scale, with emphasis instead generally being placed upon illustrating the principles of the present invention. Further, some features may be exaggerated to show details of particular components.

In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

FIGS. 1A and 1B show a single-stage chemostat vessel employed in the methods according to some embodiments of the present invention.

FIG. 2 shows a double flask apparatus wherein the black arrow identifies where a 0.22 μm filter is fitted, effectively keeping the contents of both bottles separate, save for molecules which are smaller than 0.22 μm that may pass freely through the filter (e.g., metabolite by-products of each culture and cell-cell signaling molecules). The whole apparatus is sterilized prior to use. The 0.22 μm filter prevents direct contact (cell-to-cell) of A. intestini and its co-culture companion strain.

### SUMMARY OF THE INVENTION

In an aspect, a method for improving bacterial viability following cryopreservation is presented, comprising:

a) combining a first bacterial species, wherein the first bacterial species is an Acidaminococcus species or a member of the Acidaminococcaceae family, with at least one of a second bacterial species to produce a bacterial mixture, wherein the first bacterial species is present in the bacterial mixture in an amount sufficient to confer cryoprotection to the at least one of the second bacterial species and wherein the member of the Acidaminococcaceae species is Succinispira mobilis;

b) culturing the bacterial mixture to produce a cultured bacterial mixture, wherein the culturing is for a period of time sufficient to confer the cryoprotection to the at least one of the second bacterial species in the cultured bacterial mixture; and

c) cryopreserving the cultured bacterial mixture to produce a cryopreserved bacterial culture; wherein the cryopreserved bacterial culture after reconstitution exhibits at least 10× increased bacterial proliferation of the at least one of the second bacterial species in a bacterial proliferation assay relative to bacterial proliferation of a cryopreserved bacterial culture after reconstitution comprising the at least one of the second bacterial species absent the first bacterial species.

In a particular embodiment, the cryopreserved bacterial culture after reconstitution exhibits at least 10×, 20×, 100×, 1,000×, 10,000×, or 100,000× increased bacterial proliferation of the at least one of the second bacterial species in a bacterial proliferation assay relative to bacterial proliferation of a cryopreserved bacterial culture after reconstitution comprising the at least one of the second bacterial species absent the first bacterial species.

In another particular embodiment, the cryopreserved bacterial culture after reconstitution exhibits at least 10× increased bacterial proliferation of the at least one of the second bacterial species in a bacterial proliferation assay relative to bacterial proliferation of a cryopreserved bacterial culture after reconstitution consisting essentially of the at least one of the second bacterial species.

In yet another embodiment, the Acidaminococcus species is Acidaminococcus intestini or Acidaminococcus fermentans.

In another particular embodiment, the amount of the first bacterial species sufficient to confer cryoprotection to the at least one of the second bacterial species in the bacterial mixture is between 10% and 50% of a total amount of bacteria in the bacterial mixture.

In another particular embodiment, a ratio of the first bacterial species to the at least one of the second bacterial species in the bacterial mixture is at least 1:10.

In another particular embodiment, the bacterial proliferation assay is a bacterial plating assay. In a more particular embodiment, the bacterial plating assay measures colony forming units per mL (cfu/mL).

In yet another particular embodiment, the at least one of the second bacterial species is cryoprotection refractive.

In a further particular embodiment, the at least one of the second bacterial species is derived from mammalian feces. In a more particular embodiment, the at least one of the second bacterial species is derived from human feces. In a still more particular embodiment, the at least one of the second bacterial species is at least one of Coprococcus comes, Dorea formicigenerans, Eubacterium contortum, Ruminococcus lactaris, Eubacterium rectale, Faecalibacterium prausnitzii, Eubacterium eligens, Ruminococcus torques, Roseburia intestinalis, Anaerostipes hadrus, Blautia luti, Ruminococcus obeum, Blautia stercoris, Dorea longicatena, Clostridium spiroforme, Eubacterium desmolans, Clostridium aerotolerans, Clostridium lactatifermentans, Eubacterium hallii, Clostridium hylemonae, Roseburia inulinivorans, Roseburia hominis, and Roseburia faecis.

In another embodiment, the cryopreserving comprises freezing and lyophilization.

In yet another embodiment, the reconstitution comprises dilution of the cryopreserved bacterial culture with a reconstitution medium at a 1:1 ratio of the cryopreserved bacterial culture and the reconstitution medium. In a more particular embodiment, the cryopreserved bacterial culture comprises a lyophilization-protectant medium. In a still more particular embodiment, the lyophilization-protectant medium comprises at least one of sucrose, Ficoll 70, and polyvinylpyrrolidone. In another particular embodiment, the cryopreserved bacterial culture comprises at least one of riboflavin, cysteine, and inulin. In another particular embodiment, the cryopreserved bacterial culture comprises a cryo-protectant medium. In a more particular embodiment, the cryo-protectant medium comprises at least one of glycerol, polyethylene glycol (PEG), and dimethyl sulfoxide (DMSO).

In another embodiment, the period of time sufficient to confer the cryoprotection to the at least one of the second bacterial species in the cultured bacterial mixture is at least 30 minutes or a least one hour. In a more particular embodiment, the period of time sufficient to confer the cryoprotection to the at least one of the second bacterial species in the cultured bacterial mixture ranges from 30 minutes to 2 hours or from 1-2 hours.

In another particular embodiment, the first bacterial species is alive.

In yet another particular embodiment, the method is performed under anaerobic conditions.

In another aspect, a method for improving bacterial viability following cryopreservation is presented, comprising:

a) combining a first bacterial species, wherein the first bacterial species is Acidaminococcus intestini or Acidaminococcus fermentans, with at least one of a second bacterial species to produce a bacterial mixture, wherein the first bacterial species is present in the bacterial mixture in an amount sufficient to confer cryoprotection to the at least one of the second bacterial species;

b) culturing the bacterial mixture to produce a cultured bacterial mixture, wherein the culturing is for a period of time sufficient to confer the cryoprotection to the at least one of the second bacterial species in the cultured bacterial mixture; and

c) cryopreserving the cultured bacterial mixture to produce a cryopreserved bacterial culture; wherein the cryopreserved bacterial culture after reconstitution exhibits at least 10× increased bacterial proliferation of the at least one of the second bacterial species in a bacterial proliferation assay relative to bacterial proliferation of a cryopreserved bacterial culture after reconstitution comprising the at least one of the second bacterial species absent the first bacterial species.

In a particular embodiment, the cryopreserved bacterial culture after reconstitution exhibits at least 10×, 20×, 100×, 1,000×, 10,000×, or 100,000× increased bacterial proliferation of the at least one of the second bacterial species in a bacterial proliferation assay relative to bacterial proliferation of a cryopreserved bacterial culture after reconstitution comprising the at least one of the second bacterial species absent the first bacterial species.

In another particular embodiment, the cryopreserved bacterial culture after reconstitution exhibits at least 10× increased bacterial proliferation of the at least one of the second bacterial species in a bacterial proliferation assay relative to bacterial proliferation of a cryopreserved bacterial culture after reconstitution consisting essentially of the at least one of the second bacterial species.

In another particular embodiment, the amount of the first bacterial species sufficient to confer cryoprotection to the at least one of the second bacterial species in the bacterial mixture is between 10% and 50% of a total amount of bacteria in the bacterial mixture.

In another particular embodiment, a ratio of the first bacterial species to the at least one of the second bacterial species in the bacterial mixture is at least 1:10.

In another particular embodiment, the bacterial proliferation assay is a bacterial plating assay. In a more particular embodiment, the bacterial plating assay measures colony forming units per mL (cfu/mL).

In yet another particular embodiment, the at least one of the second bacterial species is cryoprotection refractive.

In a further particular embodiment, the at least one of the second bacterial species is derived from mammalian feces. In a more particular embodiment, the at least one of the second bacterial species is derived from human feces. In a still more particular embodiment, the at least one of the second bacterial species is at least one of Coprococcus comes, Dorea formicigenerans, Eubacterium contortum, Ruminococcus lactaris, Eubacterium rectale, Faecalibacterium prausnitzii, Eubacterium eligens, Ruminococcus torques, Roseburia intestinalis, Anaerostipes hadrus, Blautia luti, Ruminococcus obeum, Blautia stercoris, Dorea longicatena, Clostridium spiroforme, Eubacterium desmolans, Clostridium aerotolerans, Clostridium lactatifermentans, Eubacterium hallii, Clostridium hylemonae, Roseburia inulinivorans, Roseburia hominis, and Roseburia faecis.

In another embodiment, the cryopreserving comprises freezing and lyophilization.

In yet another embodiment, the reconstitution comprises dilution of the cryopreserved bacterial culture with a reconstitution medium at a 1:1 ratio of the cryopreserved bacterial culture and the reconstitution medium. In a more particular embodiment, the cryopreserved bacterial culture comprises a lyophilization-protectant medium. In a still more particular embodiment, the lyophilization-protectant medium comprises at least one of sucrose, Ficoll 70, and polyvinylpyrrolidone. In another particular embodiment, the cryopreserved bacterial culture comprises at least one of riboflavin, cysteine, and inulin. In another particular embodiment, the cryopreserved bacterial culture comprises a cryo-protectant medium. In a more particular embodiment, the cryo-protectant medium comprises at least one of glycerol, polyethylene glycol (PEG), and dimethyl sulfoxide (DMSO).

In another embodiment, the period of time sufficient to confer the cryoprotection to the at least one of the second bacterial species in the cultured bacterial mixture is at least 30 minutes or a least one hour. In a more particular embodiment, the period of time sufficient to confer the cryoprotection to the at least one of the second bacterial species in the cultured bacterial mixture ranges from 30 minutes to 2 hours or from 1-2 hours.

In another particular embodiment, the first bacterial species is alive.

In yet another particular embodiment, the method is performed under anaerobic conditions.

In another aspect, an Acidaminococcus species is presented for use in a cryopreservation formulation, wherein the Acidaminococcus species improves bacterial viability of other bacterial species with which it is present in the cryopreservation formulation following reconstitution.

In another aspect, a composition comprising a cryopreservation formulation is presented, comprising:
a mixture of bacterial species in a manmade cryopreservation medium, the mixture comprising
   a) a first bacterial species, wherein the first bacterial species is Acidaminococcus intestini or Acidaminococcus fermentans; and
   b) at least one of a second bacterial species,
wherein the first bacterial species is present in the cryopreservation formulation in an amount sufficient to confer cryoprotection to the at least one of the second bacterial species upon reconstitution of the manmade cryopreservation formulation, and wherein the manmade cryopreservation formulation after reconstitution exhibits at least 10× increased bacterial proliferation of the at least one of the second bacterial species in a bacterial proliferation assay relative to bacterial proliferation of a manmade cryopreservation formulation after reconstitution comprising the at least one of the second bacterial species absent the first bacterial species.

In an embodiment of the composition, the cryopreserved bacterial culture after reconstitution exhibits at least 10× increased bacterial proliferation of the at least one of the second bacterial species in a bacterial proliferation assay relative to bacterial proliferation of a cryopreserved bacterial culture after reconstitution consisting essentially of the at least one of the second bacterial species.

In another embodiment of the composition, the amount of the first bacterial species sufficient to confer cryoprotection to the at least one of the second bacterial species in the bacterial mixture is between 10% and 50% of a total amount of bacteria in the manmade cryopreservation formulation.

In yet another embodiment of the composition, the bacterial proliferation assay is a bacterial plating assay. In a further embodiment of the composition, the bacterial plating assay measures colony forming units per mL (cfu/mL).

In another embodiment of the composition, the at least one of the second bacterial species is cryoprotection refractive.

In a further embodiment of the composition, the at least one of the second bacterial species is derived from mammalian feces. In a still further embodiment of the composition, the bacterial species is derived from human feces. In a more particular embodiment of the composition, the at least one of the second bacterial species is at least one of Coprococcus comes, Dorea formicigenerans, Eubacterium contortum, Ruminococcus lactaris, Eubacterium rectale, Faecalibacterium prausnitzii, Eubacterium eligens, Ruminococcus torques, Roseburia intestinalis, Anaerostipes hadrus, Blautia luti, Ruminococcus obeum, Blautia stercoris, Dorea longicatena, Clostridium spiroforme, Eubacterium desmolans, Clostridium aerotolerans, Clostridium lactatifermentans, Eubacterium hallii, Clostridium hylemonae, Roseburia inulinivorans, Roseburia hominis, and Roseburia faecis.

In another embodiment of the composition, the manmade cryopreservation medium comprises cryopreservation agents.

In yet another embodiment of the composition, the reconstitution comprises dilution of the cryopreservation formulation with a reconstitution medium at a 1:1 ratio of the cryopreservation formulation and the reconstitution medium.

In a further embodiment of the composition, the manmade cryopreservation medium comprises a lyophilization-protectant medium. In a particular embodiment of the composition, the lyophilization-protectant medium comprises at least one of sucrose, Ficoll 70, and polyvinylpyrrolidone. In another particular embodiment of the composition, the manmade cryopreservation medium comprises at least one of riboflavin, cysteine, and inulin. In yet another embodiment of the composition, the manmade cryopreserved bacterial culture comprises a cryo-protectant medium. In a more particular embodiment of the composition, the cryo-protectant medium comprises at least one of glycerol, polyethylene glycol (PEG), and dimethyl sulfoxide (DMSO).

In another embodiment of the composition, the first bacterial species is alive.

In another embodiment of the composition, the at least one of the second bacterial species is present in a therapeutically effective amount.

In another embodiment, the composition further comprises a pharmaceutically acceptable excipient.

In another aspect, a pharmaceutical composition comprising a cryopreservation formulation is presented, comprising:
a mixture of bacterial species in a manmade cryopreservation medium, the mixture comprising
   a) a first bacterial species, wherein the first bacterial species is Acidaminococcus intestini or Acidaminococcus fermentans; and
   b) at least one of a second bacterial species, wherein the at least one of the second bacterial species is present in a therapeutically effective amount, and
wherein the first bacterial species is present in the cryopreservation formulation in an amount sufficient to confer cryoprotection to the at least one of the second bacterial species upon reconstitution of the manmade cryopreservation formulation, and wherein the manmade cryopreservation formulation after reconstitution exhibits at least 10× increased bacterial proliferation of the at least one of the second bacterial species in a bacterial proliferation assay relative to bacterial proliferation of a manmade cryopreservation formulation after reconstitution comprising the at least one of the second bacterial species absent the first bacterial species; and a pharmaceutically acceptable excipient.

In another aspect, a method for ameliorating symptoms of a gastrointestinal disease in a subject afflicted with the gastrointestinal disease is presented, the method comprising administering the pharmaceutical composition comprising a cryopreservation formulation to the subject. In an embodiment thereof, the gastrointestinal disease comprises at least one of dysbiosis of a gastrointestinal tract, a Clostridium difficile (Clostridioides difficile) infection, and inflammatory bowel disease, irritable bowel syndrome, and diverticular disease. In an more embodiment thereof, the inflammatory bowel disease is at least one of Crohn's disease and ulcerative colitis.

In another aspect, a method is presented, comprising:
obtaining a first bacterial species;
   wherein the first bacterial species is Acidaminococcus intestini or Acidaminococcus fermentans
obtaining a second bacterial species;
combining a sufficient amount of the first bacterial species and a sufficient amount of the second bacterial species to produce a bacterial mixture;
   wherein the bacterial mixture comprises between 10% and 50% Acidaminococcus intestini of a total amount of bacteria in the bacterial mixture,
culturing the bacterial mixture for a period of time to result in a cultured mixture; and storing the cultured mixture to result in a cryopreserved bacterial culture;
wherein, when the cryopreserved bacterial culture is reconstituted, the reconstituted cryopreserved bacterial culture has at least 10× increased bacterial growth measured in colony forming units per mL (cfu/mL) of the second bacterial species compared to a reconstituted bacterial stock consisting essentially of the second bacterial species.

In some embodiments, the second bacterial species is derived from mammalian feces. In some embodiments, the second bacterial species is derived from human feces.

In some embodiments, the method further comprises lyophilizing the prepared cultured mixture. In some embodiments, the method further comprises adding a lyophilization-protectant medium. In some embodiments, the method further comprises freezing the prepared cultured mixture. In some embodiments, the method further comprises adding a cryo-protectant medium.

In some embodiments, the second bacterial species comprises: Coprococcus comes, Dorea formicigenerans, Eubacterium contortum, Ruminococcus lactaris, Eubacterium rectale, Faecalibacterium prausnitzii, Eubacterium eligens, Ruminococcus torques, Roseburia intestinalis, Anaerostipes hadrus, Blautia luti, Ruminococcus obeum, Blautia stercoris, Dorea longicatena, Clostridium spiroforme, Eubacterium desmolans, Clostridium aerotolerans, Clostridium lactatifermentans, Eubacterium hallii, Clostridium hylemonae, Roseburia inulinivorans, Roseburia hominis, Roseburia faecis, or any combination thereof.

In some embodiments, the culturing is at least 30 minutes. In some embodiments, the culturing is from 30 minutes to 2 hours. In some embodiments, the culturing is from 1 hour to 2 hours. In some embodiments, the culturing is at least 1 hour.

In some embodiments, the Acidaminococcus intestini or Acidaminococcus fermentans is live.

In some embodiments, the storing comprises adding a solution of riboflavin, cysteine, inulin, or any combination thereof.

In some embodiments, the bacterial mixture comprises between 10% and 50% Acidaminococcus intestini or Acidaminococcus fermentans of a total amount of bacteria in the bacterial mixture.

In some embodiments, the present invention provides a composition, comprising:
a stored bacterial mixture, comprising:
   a sufficient amount of a first bacterial species;
      wherein the first bacterial species is Acidaminococcus intestini or Acidaminococcus fermentans;
   a sufficient amount of a second bacterial species;
wherein, when the stored bacterial mixture is reconstituted, the reconstituted stored bacterial mixture has at least 10× increased bacterial growth measured in colony forming units per mL (cfu/mL) compared to a reconstituted bacterial stock consisting essentially of the second bacterial species.

In some embodiments, the second bacterial species comprises: Coprococcus comes, Dorea formicigenerans, Eubacterium contortum, Ruminococcus lactaris, Eubacterium rectale, Faecalibacterium prausnitzii, Eubacterium eligens, Ruminococcus torques, Roseburia intestinalis, Anaerostipes hadrus, Blautia luti, Ruminococcus obeum, Blautia stercoris, Dorea longicatena, Clostridium spiroforme, Eubacterium desmolans, Clostridium aerotolerans, Clostridium lactatifermentans, Eubacterium hallii, Clostridium hylemonae, Roseburia inulinivorans, Roseburia hominis, Roseburia faecis, or any combination thereof.

In some embodiments, the Acidaminococcus intestini is live. In some embodiments, the second bacterial species is live.

### DETAILED DESCRIPTION OF THE INVENTION

For clarity of disclosure, and not by way of limitation, the detailed description of the invention is divided into the following subsections that describe or illustrate certain features, embodiments or applications of the present invention.

Several microbes derived from human feces do not grow or show significantly reduced growth after exposure to freezing and lyophilization conditions. These microbes showed increased growth and survivability after freezing and lyophilization conditions when co-cultured with Acidaminococcus intestini (14 LG) (“A. intestini”) or Acidaminococcus fermentans (DSM 20731) (“A. fermentans”). Without being bound by theory, the mechanism behind the protective nature of A. intestini or A. fermentans may be due primarily to physical interactions occurring among the microbes, rather than microbial metabolites or agents and effects thereof.

Acidaminococcus is a genus in the phylum of Firmicutes (bacteria). The Acidaminococcus genus comprises two species: A. intestini and A. fermentans. These species are anaerobic diplococci that can use amino acids as the sole energy source for growth. They are gram-negative. They are closely related to Acidaminococcaceae type species (e.g., Succinispira mobilis) as determined by the All Species Living Tree (16S rRNA-based phylogenetic tree).

It is noteworthy that A. fermentans, in particular, is not common in human populations.

### Definitions

As used herein, the singular forms “a”, “an” and “the” include plural forms unless the content clearly dictates otherwise. Where aspects or embodiments are described in terms of Markush groups or other grouping alternatives, those skilled in the art will recognize that the invention is also thereby described in terms of any individual member or subgroup of members of the group.

As used herein, the terms “comprising”, “including”, “having” and grammatical variants thereof are to be taken as specifying the stated features, steps or components but do not preclude the addition of one or more additional features, steps, components or groups thereof.

As used herein, the term “consisting essentially of” refers to the stated features, steps or components and may further include additional elements, but only if those additional elements do not materially affect the basic characteristics of the stated features, steps or components.

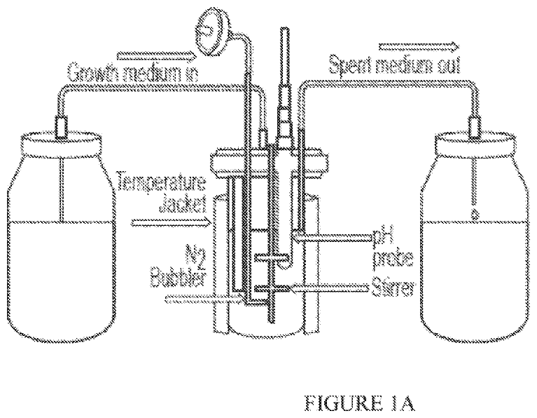

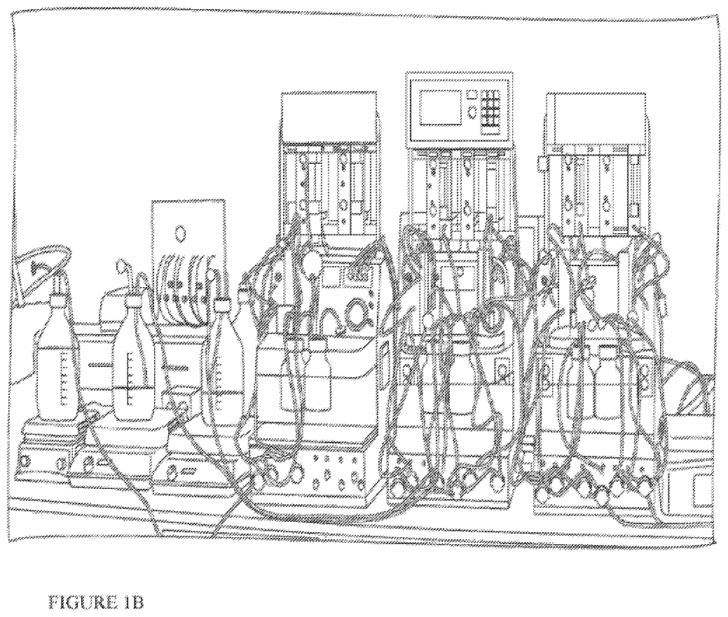

As used herein, the term “culture” or “culturing” refers to a method of multiplying microbial organisms by allowing the microbial organisms to reproduce in predetermined culture media under controlled laboratory conditions. In some embodiments, the media is generated using the apparatus shown in FIGS. 1A and 1B and uses the methods described in US published application no. 20140363397 or US published application no. 20140342438.

As used herein, the term “lyophilization” refers to a process in which a composition is first frozen and then, while still in the frozen state, undergoes sublimation and desorption to reduce the major portion of the water and solvent in the composition, with the intent to limit biological and chemical reactions at the designated storage temperature for short, medium, or long term preservation

As used herein, the term “neat dilution” refers to an undiluted culture which is typically plated or grown in culture.

As used herein, the term “reconstitute” or “reconstituting” refers to a method of reanimating frozen and/or dried microbial organisms which involves dilution in a suitable reconstitution medium to produce a reconstituted composition of live microbial organisms. Exemplary reconstitution media include, without limitation, 1× phosphate buffered saline (PBS) or a similar physiological salt solution which preserves viability, bacterial culture media suited to the bacteria undergoing reconstitution. Other reconstitution media include tryptic soy broth with supplemented hemin and menadione, brain-heart infusion broth, Wilkins-Chalgren broth and fastidious anaerobe broth.

When a numerical value is preceded by the term “about”, the term “about” is intended to indicate +/−10%.

As used herein, “anaerobic bacteria” refers to bacteria which are facultatively anaerobic as well as bacteria which are strictly anaerobic.

As used herein, “standard culture media” refers to common and/or commercially available growth media for microorganisms, such as nutrient broths and agar plates, of which many variations are known in the art. Standard culture media generally contains at least a carbon source for bacterial growth, e.g., a sugar such as glucose; various salts which are required for bacterial growth, e.g., magnesium, nitrogen, phosphorus, and/or sulfur; and water. Non-limiting examples of standard culture media include Luris Bertani (LB) media, Al broth, and culture media described herein. Standard culture media for use in methods provided herein will be selected by a skilled artisan based on common general knowledge. The terms “standard culture media” and “standard laboratory culture media” are used interchangeably herein.

As used herein, the terms “pure isolate,” “single isolate” and “isolate” are used interchangeably to refer to a culture comprising a single bacterial species or strain, e.g., grown axenically, in isolation from other bacterial species or strains.

For strains listed in the tables herein, the closest bacterial species was determined using the 16S rRNA full length sequences, which were aligned with the NAST server and were then classified using the GreenGenes classification server.

### Feces Collection and Bacterial Processing Therefrom

The donor is asked to void feces in a private bathroom near the lab, into a provided sterile pot. The pot is immediately transported to the lab and placed into an anaerobic container within 5 minutes of voiding. It is noted that some of the isolates, in particular Roseburia spp., are extremely sensitive to oxygen, and thus it is critical that the voided sample is protected from exposure to oxygen even for the short-term (5 mins).

Once in the anaerobic chamber, a 10 g sample of feces is weighed into 50 mL sterile, pre-reduced saline and placed into a sterile stomacher bag, which is placed into the stomacher instrument and pummeled for 2 minutes to homogenize the sample. The homogenate is then placed into a sterile centrifuge tube and spun at low speed to sediment large particles, thereby producing a processed sample essentially free of large particle sediment.

Two rounds of microbial isolation may then performed as follows: a dilution series of the homogenate supernatant is made in sterile, pre-reduced saline. 100 uL of each dilution is separately plated onto quadruplicates of prepared agar media as below:

Fastidious anaerobe agar (Lab 90) supplemented
with 5% defibrinated sheep blood;
Fastidious anaerobe agar without blood supplementation;
Fastidious anaerobe agar+5% defibrinated sheep blood+3% ‘liquid gold’ (described below);
Fastidious anaerobe agar+3% liquid gold;
deMan-Rogosa-Sharpe (MRS) media (purchased from Oxoid Limited, Hampshire, United Kingdom), enriches for Lactobacillus and Bifidobacterium spp.);
Mucin agar formulated in-house (minimal media with mucin as the only carbon source; this is used since some bacterial species of the human gut micro flora are known to utilize mucin as a carbon source); and
LS agar, which is agar supplemented with 3% v/v spent cell culture supernatant taken from a confluent culture of LS174 T cells (a human colonic cell line which secretes mucin; available from the ATCC).

Selection for microbes may optionally also include a screening step to identify microbes that sporulate. Such screening is typically performed by exposing the microbial population to an ethanol shock. To this end, a homogenate sample of microbes is exposed to 100% ethanol for 20 mins to 1 hr, then the microbes are spun down and washed twice with PBS, and then plated as described below. This is an extra step that is performed with some of the homogenate sample. It selects for sporulating microbes, since endospores are resistant to ethanol, whereas actively growing cells are not.

Cell culture media may be prepared from: 1 package of minimum essential medium (Gibco #41500-034); 2.2 g sodium bicarbonate (Sigma); 4.766 g HEPES buffer (Sigma); 10 mL 100 mM sodium pyruvate solution; 10% (v/v) heat inactivated fetal bovine serum (Gibco) (30 min. at 56° C.), brought up to 1 liter in double-distilled water and filter-sterilized through a 0.22 μm pore-sized filter (Millipore). Spent cell culture medium is medium taken from the supernatant of LS174T cells cultured at 37° C. in 5% C02 for 5 days and filtered through a 0.22 μm pore-sized filter to remove host cells. This medium is used since some bacterial isolates may require human cell signals for proliferation and growth in vitro.

Plates are typically incubated for 2 weeks in a humidified anaerobe chamber (Bug Box from Ruskinn, Bridegend, United Kingdom), and inspected for growth every few days. Isolated colonies are picked to new plates and allowed to grow for the same length of time, to ensure that pure cultures are obtained; any second or third colony type which grow are removed. In a particular embodiment, cultures may be carefully cryopreserved in freezing media comprising a milk-glycerol-dimethyl sulfoxide mix designed for preservation of anaerobes, containing 60 g Carnation skim milk powder (Zehr's), 5 mL DMSO (Sigma) and 5 mL glycerol (Sigma) and double distilled H₂0 to bring total volume to 500 mL.

Once strains are isolated, optimal growth conditions are determined empirically by culturing each isolate on each different medium type as above, and determining which media gives the best growth. It is important to note that the strains are kept in an anaerobic environment at all times. They are never worked with outside of an anaerobic environment, e.g., the present inventors never worked with the live bacteria on an open bench and the microbes are kept as healthy as possible at all times.

For the second round of characterization, a chemostat may be used to first stabilize the microbial community as a whole, in vitro. Steady state (equilibrium) is reached after about 1 month, following which the dilution and plating methods as above are used to try to isolate further microorganisms. The chemostat is used to effectively sample and culture the community and also to enrich for some gut microbes that may have been present in only small numbers in the original fecal sample. These organisms may be, for example, microbes that are intimately associated with the mucosa and are ‘sloughed off’ along with dead cells in the colon. The chemostat environment allows some of these bugs to survive and proliferate effectively, enriching their numbers so they can be plate-cultured as above.

The terms “cultured” and “grown” are sometimes used interchangeably herein.

### Single-Stage Chemostats and Inoculation

An exemplary protocol for isolating bacteria from the human distal gut is presented below. The present inventors developed a single-stage chemostat vessel to model the human distal gut microbiota by modifying a Multifors fermentation system (Infors, Switzerland) as described in US published application no. 20140342438. Conversion from a fermentation system into a chemostat was accomplished by blocking off the condenser and bubbling nitrogen gas through the culture. The pressure build up forced the waste out of a metal tube (formerly a sampling tube) at a set height and allowed for the maintenance of a 400 mL working volume.

Throughout the duration of the experiment, the vessels were kept anaerobic by bubbling filtered nitrogen gas (Praxair) through the culture. Temperature (37° C.) and pH (set to 7.0; usually fluctuated around 6.9 to 7 in the culture) were automatically controlled and maintained by a computer-operated system. The system maintained the culture pH using 5% (v/v) HCl (Sigma) and 5% (w/v) NaOH (Sigma). The growth medium was continuously fed into the vessel at a rate of 400 mL/day (16.7 mL/hour) to give a retention time of 24 hours, a value set to mimic the retention time of the distal gut. Another retention time of 65 hours (−148 mL/day, 6.2 mL/hour) was also tested to determine the effect of retention time on the composition of the chemostat community.

Since the growth medium contained components which cannot survive sterilization by autoclaving, the vessels were autoclaved with 400 mL of ddH₂0. During autoclaving, the waste pipes were adjusted so the metal tube reached the bottom of the vessel. Once the vessel cooled it was fitted to the rest of the computer operated unit, filtered nitrogen gas was bubbled through the water to pressurize and drain the vessel. The waste pipe was then raised to the working volume (400 mL) and 300 mL of sterile media was pumped into the vessel. The vessel was then left stirring, heating, and degassing overnight. To check for contamination within the vessel, each vessel was aseptically sampled and plated out (both aerobically and anaerobically) on fastidious anaerobe agar (FAA) supplemented with 5% defibrinated sheep blood. This procedure was repeated one day before inoculation and immediately prior to inoculation to ensure contamination was avoided.

### Collection and Preparation of Fecal Inocula

Fresh fecal samples can be isolated from a variety of human donors, ranging from healthy female or male donors (e.g., with no history of antibiotic use in the 10 years prior to stool donation to individuals with known disorders/diseases). Research Ethics Board (REB) approval is obtained for fecal collection and use in these experiments.

To prepare the inoculum, freshly voided stool samples are collected and immediately placed in an anaerobic chamber (in an atmosphere of 90% N₂, 5% C0₂ and 5% H₂). A 10% (w/v) fecal slurry is immediately prepared by macerating 5 g of fresh feces in 50 mL of anaerobic phosphate buffered saline (PBS) for 1 minute using a stomacher (Tekmar Stomacher Lab Blender, made by Seward). The resulting fecal slurry is centrifuged for 10 minutes at 1500 rpm to remove large food residues. The resulting supernatant may be used as an inoculum.

### Inoculation Process

To give a final working volume of 400 mL, 100 mL of, e.g., 10% inocula is added to the 300 mL of sterile media in each vessel. Immediately following inoculation, the pH controls are turned on so the vessel pH is adjusted to and maintained at a pH of about 6.9 to 7.0. During the first 24 hours post-inoculation the communities are grown in batch culture to allow the community to adjust from in vivo to in vitro conditions and avoid culture washout. During this period the vessels are heated, degassed and stirred with continuous pH adjustment. After this 24 hour period the feed pumps are turned on and the vessels run as chemostats. Fresh culture medium is added continuously and waste continuously removed.

In the chemostat, culture conditions and media supply are maintained constant. The chemostat system is generally set with a retention time of 24 hours to mimic distal gut transit time.

### Preparation of the Growth Medium

The culture medium may be prepared in the following steps (for 2 L):

Mixture 1:

The following reagents were dissolved in 1800 mL of distilled water (ddH20): peptone water, 4 g (Oxoid Limited); Yeast extract, 4 g (Oxoid Limited); NaHCO₃, 4 g (Sigma); CaC12, 0.02 g (Sigma); Pectin (from citrus), 4 g (Sigma); Xylan (from beechwood), 4 g (Sigma); Arabinogalactan, 4 g (Sigma); Starch (from wheat, unmodified), 10 g (Sigma); Casein, 6 g (Sigma); inulin (from Dahlia tubers), 2 g (Sigma); NaCl, 0.2 g (Sigma). Water (ddH₂0) was added to 1900 mL, as the volume is reduced to 1800 mL after autoclaving. The mixture was sterilized by autoclaving at 121° C. for 60 min and allowed to cool overnight.

Mixture 2:

The following reagents (all purchased from Sigma) were dissolved in 100 mL of distilled water (Mixture 2A): K₂HP0₄, 0.08 g; KH₂P0₄, 0.08 g; MgS0₄, 0.02 g; Hemin, 0.01 g; Menadione, 0.002 g. Bile salts (1 g) was dissolved in 50 mL of distilled water (Mixture 2B). L-cysteine HCl (1 g) was also dissolved in 50 mL of distilled water (Mixture 2C). After Mixtures 2B and 2C dissolved they were added to Mixture 2A resulting in the formation of a fine white precipitate. This precipitate was then dissolved by the drop-wise addition of 6M KOH until a clear, brown solution was formed (Mixture 2). This mixture (200 mL total volume) was sterilized by filtering through a 0.22 μm filter.

Culture media (“Media 1”): Mixture 2 (0.2 L) is aseptically added to mixture 1 (1.8 L), in order to reach the final volume of 2 L. To prevent future foaming, 5 mL of antifoam B silicone emulsion (J. T. Baker) was aseptically added to each 2 L bottle of media. The media was stored at 4° C. until use for a maximum of two weeks. A bit of media was plated out on FAA (aerobically and anaerobically) the day before adding to chemostat and immediately after taking off the chemostat, to check for contamination.

The media was pumped into each vessel using a peristaltic pump whose speed is controlled by the computer operated system. To pump media from the bottles into the vessel, standard GL-45 glass bottle lids (VWR) had holes drilled into them to fit two stainless steel metal tubes. When Mixture 1 is prepared, the media bottle had all the required silicone tubing and 0.22 μm filters attached.

Each vessel was fed from one media bottle with a 2 L volume of media. Since the tubing which supplied the media to the vessel is also changed as each media bottle is changed, this helps to prevent back-growth of bacteria from the vessel into the sterile media reservoir. Each media bottle is plated out on supplemented FAA and grown both aerobically and anaerobically before each bottle is added to the chemostat and after each bottle is removed from the chemostat.

As used herein, the term “cryopreservation agents” refers to agents that reduce or prevent the formation of ice crystals and/or protect bacterial cells from increased solute concentration (caused by the formation of ice). Common cryoprotectants include dimethylsulfoxide, skim milk, and complex sugars.

As used herein, the terms “cryoprotection refractive” or “sensitive to cryoprotection” refer to organisms which, even in the presence of cryoprotectants, are still fragile enough that they suffer considerable damage during the freezing process, thereby impeding their survival under freezing conditions.

As used herein, the term “cryoprotection” refers to the use of super-cold temperatures (<70° C.) to freeze microbial cells and hold them in a state of suspended animation.

As used herein, the term “bacterial proliferation assay” refers to method/s used to determine viability of a microbe before and after cryopreservation. Cell growth is quantified before and after cryopreservation using either dilution series and direct plate counts on agar, or by flow cytometry with specific staining for live vs. dead cells, using, for example, propidium iodide.

As used herein, the term “cryopreserving” refers to the act of freezing a microbial culture with the intent of maintaining as much viability as possible during storage.

As used herein, the term “cryopreserved bacterial culture” refers to bacterial cells which have been treated with cryoprotectants and stored at optimal temperatures (<70° C.).

### Method for Storing Bacteria

In some embodiments, the present invention provides a method, comprising:
obtaining a first bacterial species;
   wherein the first bacterial species is an Acidaminococcus species (e.g., Acidaminococcus intestini or Acidaminococcus fermentans) or an Acidaminococcaceae type species (e.g., Succinispira mobilis),
obtaining a second bacterial species;
combining a sufficient amount of the first bacterial species and a sufficient amount of the second bacterial species to produce a bacterial mixture;
   wherein the bacterial mixture comprises between 10% and 50% Acidaminococcus species (e.g., Acidaminococcus intestini or Acidaminococcus fermentans) or an Acidaminococcaceae type species (e.g., Succinispira mobilis) of a total amount of bacteria in the bacterial mixture,
culturing the bacterial mixture for a period of time to result in a cultured mixture; and storing the cultured mixture to result in a cryopreserved bacterial culture;
wherein, when the cryopreserved bacterial culture is reconstituted, the reconstituted cryopreserved bacterial culture has at least 10× increased bacterial growth measured in colony forming units per mL (cfu/mL) of the second bacterial species compared to a reconstituted bacterial stock consisting essentially of the second bacterial species.

In some embodiments, the second bacterial species is derived from mammalian feces. In some embodiments, the second bacterial species is derived from human feces.

In some embodiments, the method further comprises lyophilizing the prepared cultured mixture. In some embodiments, the method further comprises adding a lyophilization-protectant medium. In some embodiments, the method further comprises freezing the prepared cultured mixture. In some embodiments, the method further comprises adding a cryo-protectant medium.

In some embodiments, the second bacterial species comprises: Coprococcus comes, Dorea formicigenerans, Eubacterium contortum, Ruminococcus lactaris, Eubacterium rectale, Faecalibacterium prausnitzii, Eubacterium eligens, Ruminococcus torques, Roseburia intestinalis, Anaerostipes hadrus, Blautia luti, Ruminococcus obeum, Blautia stercoris, Dorea longicatena, Clostridium spiroforme, Eubacterium desmolans, Clostridium aerotolerans, Clostridium lactatifermentans, Eubacterium hallii, Clostridium hylemonae, Roseburia inulinivorans, Roseburia hominis, Roseburia faecis, or any combination thereof.

In some embodiments, the culturing is at least 30 minutes. In some embodiments, the culturing is from 30 minutes to 2 hours. In some embodiments, the culturing is from 1 hour to 2 hours. In some embodiments, the culturing is at least 1 hour.

In some embodiments, the Acidaminococcus species (e.g., Acidaminococcus intestini or Acidaminococcus fermentans) or an Acidaminococcaceae type species (e.g., Succinispira mobilis) is live.

In some embodiments, the storing comprises adding a solution of riboflavin, cysteine, inulin, or any combination thereof.

In some embodiments, the bacterial mixture comprises between 10% and 50% Acidaminococcus species (e.g., Acidaminococcus intestini or Acidaminococcus fermentans) or an Acidaminococcaceae type species (e.g., Succinispira mobilis) of a total amount of bacteria in the bacterial mixture.

### Obtaining a First Bacterial Species

In some embodiments, the Acidaminococcus species (e.g., Acidaminococcus intestini or Acidaminococcus fermentans) or an Acidaminococcaceae type species (e.g., Succinispira mobilis) is live.

In some embodiments, the Acidaminococcus intestini comprises Acidaminococcus intestini (14 LG), Acidaminococcus intestini (GAM7), Acidaminococcus intestini (CC1/6 D9), or any combination thereof. In some embodiments, the Acidaminococcus intestini comprises Acidaminococcus intestini (RyC-MR95), Acidaminococcus intestini (DNF00404), Acidaminococcus intestini (ADV 255.99), Acidaminococcus intestini (DSM 21505), or any combination thereof.

In some embodiments, the Acidaminococcus fermentans comprises Acidaminococcus fermentans (DSM 20731), Acidaminococcus fermentans Rogosa (VR4; available for purchase from the ATCC®25085™),), Acidaminococcus fermentans (RYC4093), Acidaminococcus fermentans (RYC4356), Acidaminococcus fermentans (RYC-MR95), or any combination thereof.

In some embodiments, the Acidaminococcaceae type species is Succinispira mobilis (DSM 6222; available for purchase from the ATCC®700845™), Succinispira mobilis (DSM 6222T), or any combination thereof.

In some embodiments, the Acidaminococcus species (e.g., Acidaminococcus intestini or Acidaminococcus fermentans) or an Acidaminococcaceae type species (e.g., Succinispira mobilis) is derived from mammalian feces. In some embodiments, the Acidaminococcus species (e.g., Acidaminococcus intestini or Acidaminococcus fermentans) or an Acidaminococcaceae type species (e.g., Succinispira mobilis) is derived from human feces. In some embodiments, the Acidaminococcus species (e.g., Acidaminococcus intestini or Acidaminococcus fermentans) or an Acidaminococcaceae type species (e.g., Succinispira mobilis) is derived from a healthy patient. In some embodiments, the Acidaminococcus species (e.g., Acidaminococcus intestini or Acidaminococcus fermentans) or an Acidaminococcaceae type species (e.g., Succinispira mobilis) is derived from a healthy patient according to the methods disclosed in U.S. Patent Application No. 20140342438, which is herein incorporated by reference in its entirety.

In some embodiments, the Acidaminococcus species (e.g., Acidaminococcus intestini or Acidaminococcus fermentans) or an Acidaminococcaceae type species (e.g., Succinispira mobilis) is derived from a patient with a gastrointestinal disease. In some embodiments, the Acidaminococcus species (e.g., Acidaminococcus intestini or Acidaminococcus fermentans) or an Acidaminococcaceae type species (e.g., Succinispira mobilis) is obtained from a patient with a gastrointestinal disease according to the methods disclosed in U.S. Patent Application No. 20140342438. In some embodiments, the Acidaminococcus species (e.g., Acidaminococcus intestini or Acidaminococcus fermentans) or an Acidaminococcaceae type species (e.g., Succinispira mobilis) is obtained from a patient with a gastrointestinal disease according to the methods disclosed in U.S. Patent Application No. 20140363397, which is herein incorporated by reference in its entirety.

In some embodiments, the gastrointestinal disease comprises dysbiosis, Clostridium difficile (Clostridioides difficile) infection, inflammatory bowel disease: Crohn's disease and ulcerative colitis, irritable bowel syndrome, and/or diverticular disease.

### Obtaining a Second Bacterial Species

In some embodiments, the at least one of a second bacterial species is derived from mammalian feces. In some embodiments, the at least one of a second bacterial species is derived from human feces. In some embodiments, the at least one of a second bacterial species is derived from a healthy patient. In some embodiments, the at least one of a second bacterial species is derived from a healthy patient according to the methods disclosed in U.S. Patent Application No. 20140342438.

In some embodiments, the at least one of a second bacterial species comprises: Coprococcus comes, Dorea formicigenerans, Eubacterium contortum, Ruminococcus lactaris, Eubacterium rectale, Faecalibacterium prausnitzii, Eubacterium eligens, Ruminococcus torques, Roseburia intestinalis, Anaerostipes hadrus, Blautia luti, Ruminococcus obeum, Blautia stercoris, Dorea longicatena, Clostridium spiroforme, Eubacterium desmolans, Clostridium aerotolerans, Clostridium lactatifermentans, Eubacterium hallii, Clostridium hylemonae, Roseburia inulinivorans, Roseburia hominis, Roseburia faecis, or any combination thereof.

In some embodiments, the at least one of a second bacterial species is derived from a patient with a gastrointestinal disease. In some embodiments, the at least one of a second bacterial species is obtained from a patient with a gastrointestinal disease according to the methods disclosed in U.S. Patent Application No. 20140342438. In some embodiments, the at least one of a second bacterial species is obtained from a patient with a gastrointestinal disease according to the methods disclosed in U.S. Patent Application No. 20140363397.

### Producing a Bacterial Mixture

In some embodiments, the bacterial mixture comprises between 0.1% and 99.9% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 1% and 99.9% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 10% and 99.9% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 20% and 99.9% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 30% and 99.9% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 40% and 99.9% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 50% and 99.9% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 60% and 99.9% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 70% and 99.9% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 80% and 99.9% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 90% and 99.9% Acidaminococcus species (e.g., Acidaminococcus intestini or Acidaminococcus fermentans) or an Acidaminococcaceae type species (e.g., Succinispira mobilis) of a total amount of bacteria in the bacterial mixture.

In some embodiments, the bacterial mixture comprises between 0.1% and 90% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 0.1% and 80% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 0.1% and 70% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 0.1% and 60% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 0.1% and 50% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 0.1% and 40% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 0.1% and 30% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 0.1% and 20% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 0.1% and 10% Acidaminococcus species (e.g., Acidaminococcus intestini or Acidaminococcus fermentans) or an Acidaminococcaceae type species (e.g., Succinispira mobilis) of a total amount of bacteria in the bacterial mixture.

In some embodiments, the bacterial mixture comprises between 10% and 90% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 10% and 80% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 10% and 70% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 10% and 60% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 10% and 50% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 10% and 40% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 10% and 30% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 10% and 20% Acidaminococcus species (e.g., Acidaminococcus intestini or Acidaminococcus fermentans) or an Acidaminococcaceae type species (e.g., Succinispira mobilis) of a total amount of bacteria in the bacterial mixture.

In some embodiments, the bacterial mixture comprises between 20% and 90% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 30% and 90% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 40% and 90% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 50% and 90% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 60% and 90% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 70% and 90% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 80% and 90% Acidaminococcus species (e.g., Acidaminococcus intestini or Acidaminococcus fermentans) or an Acidaminococcaceae type species (e.g., Succinispira mobilis) of a total amount of bacteria in the bacterial mixture.

In some embodiments, the bacterial mixture comprises between 30% and 80% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 40% and 70% Acidaminococcus species (e.g., Acidaminococcus intestini or Acidaminococcus fermentans) or an Acidaminococcaceae type species (e.g., Succinispira mobilis) of a total amount of bacteria in the bacterial mixture. In some embodiments, the bacterial mixture comprises between 50% and 60% Acidaminococcus species (e.g., Acidaminococcus intestini or Acidaminococcus fermentans) or an Acidaminococcaceae type species (e.g., Succinispira mobilis) of a total amount of bacteria in the bacterial mixture.

In some embodiments, the bacterial mixture comprises between 0.1% and 99.9% of the at least one of a second bacterial species of a total amount of bacteria in the bacterial mixture. In some embodiments, the bacterial mixture comprises between 1% and 99.9% of the at least one of a second bacterial species of a total amount of bacteria in the bacterial mixture. In some embodiments, the bacterial mixture comprises between 10% and 99.9% the at least one of a second bacterial species of a total amount of bacteria in the bacterial mixture. In some embodiments, the bacterial mixture comprises between 20% and 99.9% of the at least one of a second bacterial species of a total amount of bacteria in the bacterial mixture. In some embodiments, the bacterial mixture comprises between 30% and 99.9% of the at least one of a second bacterial species of a total amount of bacteria in the bacterial mixture. In some embodiments, the bacterial mixture comprises between 40% and 99.9% of the at least one of a second bacterial species of a total amount of bacteria in the bacterial mixture. In some embodiments, the bacterial mixture comprises between 50% and 99.9% of the at least one of a second bacterial species of a total amount of bacteria in the bacterial mixture. In some embodiments, the bacterial mixture comprises between 60% and 99.9% of the at least one of a second bacterial species of a total amount of bacteria in the bacterial mixture. In some embodiments, the bacterial mixture comprises between 70% and 99.9% of the at least one of a second bacterial species of a total amount of bacteria in the bacterial mixture. In some embodiments, the bacterial mixture comprises between 80% and 99.9% of the at least one of a second bacterial species of a total amount of bacteria in the bacterial mixture. In some embodiments, the bacterial mixture comprises between 90% and 99.9% of the at least one of a second bacterial species of a total amount of bacteria in the bacterial mixture.

In some embodiments, the bacterial mixture comprises between 0.1% and 90% of the at least one of a second bacterial species of a total amount of bacteria in the bacterial mixture. In some embodiments, the bacterial mixture comprises between 0.1% and 80% of the at least one of a second bacterial species of a total amount of bacteria in the bacterial mixture. In some embodiments, the bacterial mixture comprises between 0.1% and 70% of the at least one of a second bacterial species of a total amount of bacteria in the bacterial mixture. In some embodiments, the bacterial mixture comprises between 0.1% and 60% of the at least one of a second bacterial species of a total amount of bacteria in the bacterial mixture. In some embodiments, the bacterial mixture comprises between 0.1% and 50% of the at least one of a second bacterial species of a total amount of bacteria in the bacterial mixture. In some embodiments, the bacterial mixture comprises between 0.1% and 40% of the at least one of a second bacterial species of a total amount of bacteria in the bacterial mixture. In some embodiments, the bacterial mixture comprises between 0.1% and 30% of the at least one of a second bacterial species of a total amount of bacteria in the bacterial mixture. In some embodiments, the bacterial mixture comprises between 0.1% and 20% of the at least one of a second bacterial species of a total amount of bacteria in the bacterial mixture. In some embodiments, the bacterial mixture comprises between 0.1% and 10% of the at least one of a second bacterial species of a total amount of bacteria in the bacterial mixture.

In some embodiments, the bacterial mixture comprises between 10% and 90% of the at least one of a second bacterial species of a total amount of bacteria in the bacterial mixture. In some embodiments, the bacterial mixture comprises between 10% and 80% of the at least one of a second bacterial species of a total amount of bacteria in the bacterial mixture. In some embodiments, the bacterial mixture comprises between 10% and 70% of the at least one of a second bacterial species of a total amount of bacteria in the bacterial mixture. In some embodiments, the bacterial mixture comprises between 10% and 60% of the at least one of a second bacterial species of a total amount of bacteria in the bacterial mixture. In some embodiments, the bacterial mixture comprises between 10% and 50% of the at least one of a second bacterial species of a total amount of bacteria in the bacterial mixture. In some embodiments, the bacterial mixture comprises between 10% and 40% of the at least one of a second bacterial species of a total amount of bacteria in the bacterial mixture. In some embodiments, the bacterial mixture comprises between 10% and 30% of the at least one of a second bacterial species of a total amount of bacteria in the bacterial mixture. In some embodiments, the bacterial mixture comprises between 10% and 20% of the at least one of a second bacterial species of a total amount of bacteria in the bacterial mixture.

In some embodiments, the bacterial mixture comprises between 20% and 90% of the at least one of a second bacterial species of a total amount of bacteria in the bacterial mixture. In some embodiments, the bacterial mixture comprises between 30% and 90% of the at least one of a second bacterial species of a total amount of bacteria in the bacterial mixture. In some embodiments, the bacterial mixture comprises between 40% and 90% of the at least one of a second bacterial species of a total amount of bacteria in the bacterial mixture. In some embodiments, the bacterial mixture comprises between 50% and 90% of the at least one of a second bacterial species of a total amount of bacteria in the bacterial mixture. In some embodiments, the bacterial mixture comprises between 60% and 90% of the at least one of a second bacterial species of a total amount of bacteria in the bacterial mixture. In some embodiments, the bacterial mixture comprises between 70% and 90% of the at least one of a second bacterial species of a total amount of bacteria in the bacterial mixture. In some embodiments, the bacterial mixture comprises between 80% and 90 of the at least one of a second bacterial species of a total amount of bacteria in the bacterial mixture.

In some embodiments, the bacterial mixture comprises between 30% and 80% of the at least one of a second bacterial species of a total amount of bacteria in the bacterial mixture. In some embodiments, the bacterial mixture comprises between 40% and 70% of the at least one of a second bacterial species of a total amount of bacteria in the bacterial mixture. In some embodiments, the bacterial mixture comprises between 50% and 60% of the at least one of a second bacterial species of a total amount of bacteria in the bacterial mixture.

In some embodiments, the bacterial mixture comprises at least Acidaminococcus species (e.g., Acidaminococcus intestini or Acidaminococcus fermentans) or an Acidaminococcaceae type species (e.g., Succinispira mobilis) and a second bacterial species. In some embodiments, the second bacterial species comprises: Coprococcus comes, Dorea formicigenerans, Eubacterium contortum, Ruminococcus lactaris, Eubacterium rectale, Faecalibacterium prausnitzii, Eubacterium eligens, Ruminococcus torques, Roseburia intestinalis, Anaerostipes hadrus, Blautia luti, Ruminococcus obeum, Blautia stercoris, Dorea longicatena, Clostridium spiroforme, Eubacterium desmolans, Clostridium aerotolerans, Clostridium lactatifermentans, Eubacterium hallii, Clostridium hylemonae, Roseburia inulinivorans, Roseburia hominis, Roseburia faecis, or any combination thereof.

### Culturing a Bacterial Mixture

In some embodiments, the culturing is at least 30 minutes. In some embodiments, the culturing is from 30 minutes to 2 hours. In some embodiments, the culturing is from 1 hour to 2 hours. In some embodiments, the culturing is at least 1 hour.

In some embodiments, the culturing can be performed for up to 48 hours. In some embodiments, the culturing is from 1 hour to 48 hours. In some embodiments, the culturing can be performed for up to 48 hours. In some embodiments, the culturing is from 2 hours to 48 hours. In some embodiments, the culturing is from 4 hours to 48 hours. In some embodiments, the culturing is from 8 hours to 48 hours. In some embodiments, the culturing is from 12 hours to 48 hours. In some embodiments, the culturing is from 24 hours to 48 hours. In some embodiments, the culturing is from 1 hour to 24 hours. In some embodiments, the culturing is from 1 hour to 12 hours. In some embodiments, the culturing is from 1 hour to 8 hours. In some embodiments, the culturing is from 1 hour to 4 hours.

In some embodiments, the method comprises culturing the bacterial mixture for a period of time to result in a cultured mixture. In some embodiments, the culturing is performed using the methods disclosed in U.S. Patent Application No. 20140342438, which is herein incorporated by reference in its entirety.

### Cryopreserved Bacterial Cultures

In some embodiments, the cryopreserved bacterial culture comprises riboflavin, cysteine, inulin, or any combination thereof.

In some embodiments, the cryopreserved bacterial culture comprises a lyophilization-protectant medium. In some embodiments, the lyophilization-protectant medium comprises sucrose, Ficoll 70, polyvinylpyrrolidone, or any combination thereof.

In some embodiments, the cryopreserved bacterial culture comprises a cryo-protectant medium. In some embodiments, the cryo-protectant medium comprises glycerol, polyethylene glycol (PEG), dimethyl sulfoxide (DMSO), or any combination thereof.

In some embodiments, the cryopreserving the cultured bacterial mixture comprises adding a suitable cryopreservation composition to the cultured bacterial mixture and freezing the composition comprising the cultured bacterial mixture and the suitable cryopreservation composition to produce a frozen bacterial cryopreservation composition. In some embodiments, the freezing is at or below 0 degrees Celsius (C). In some embodiments, the freezing is at or below −20 degrees C. In some embodiments, the freezing is at or below −60 degrees C. In some embodiments, the freezing is at or below −80 degrees C.

In some embodiments, the freezing is at or below 0 degrees Celsius (C). In some embodiments, the freezing is at or below −20 degrees C. In some embodiments, the freezing is at or below −60 degrees C. In some embodiments, the freezing is at or below −80 degrees C. In some embodiments, the freezing is from −100 to 0 degrees C. In some embodiments, the freezing is from −80 to 0 degrees C. In some embodiments, the freezing is from −60 to 0 degrees C. In some embodiments, the freezing is from −40 to 0 degrees C. In some embodiments, the freezing is from −20 to 0 degrees C. In some embodiments, the freezing is from −100 to −20 degrees C. In some embodiments, the freezing is from −100 to −40 degrees C. In some embodiments, the freezing is from −100 to −60 degrees C. In some embodiments, the freezing is from −100 to −80 degrees C. In some embodiments, the freezing is from −80 to −20 degrees C. In some embodiments, the freezing is from −60 to −40 degrees C.

In some embodiments, the cryopreserving the cultured bacterial mixture comprises adding a suitable cryopreservation composition to the cultured bacterial mixture, freezing the composition comprising the cultured bacterial mixture and the suitable cryopreservation composition to produce a frozen bacterial cryopreservation composition, and lyophilizing the frozen bacterial cryopreservation composition to produce a cryopreserved bacterial culture. In some embodiments, the lyophilizing is performed using typically used methods known to a person having ordinary skill in the art.

In some embodiments, preserving the cultured bacterial mixture to produce a preserved bacterial culture comprises adding a suitable preservation composition to the cultured bacterial mixture and lyophilizing the composition comprising the cultured bacterial mixture and the suitable preservation composition to produce a dehydrated, preserved bacterial culture. In some embodiments, the lyophilizing is performed using typically used methods known to a person having ordinary skill in the art.

### Reconstituting a Cryopreserved Bacterial Culture

In some embodiments, the reconstituting of a cryopreserved bacterial culture can be performed using methods known in the art for frozen or frozen and lyophilized (freeze-dried) cultures. As a non-limiting example, for reconstituting a freeze-dried culture, a suitable volume of medium can be used to rehydrate a bacterial species for streaking, growth in a culture tube, etc. As a further non-limiting example, for reconstituting a frozen culture, a portion of the frozen culture can be defrosted and used to inoculate a plate, a culture, etc. In some embodiments, the medium can be generated using the methods disclosed in U.S. Patent Application No. 20140342438.

In some embodiments, when the cryopreserved bacterial culture is reconstituted, the reconstituted cryopreserved bacterial culture has at least 10× increased bacterial growth measured in colony forming units per mL (cfu/mL) of the at least one of a second bacterial species compared to a reconstituted bacterial stock consisting essentially of the at least one of a second bacterial species. In some embodiments, when the cryopreserved bacterial culture is reconstituted, the cryopreserved bacterial culture has at least 100× increased bacterial growth measured in colony forming units per mL (cfu/mL) of the at least one of a second bacterial species compared to a reconstituted bacterial stock consisting essentially of the at least one of a second bacterial species. In some embodiments, when the cryopreserved bacterial culture is reconstituted, the reconstituted cryopreserved bacterial culture has at least 1,000× increased bacterial growth measured in colony forming units per mL (cfu/mL) of the at least one of a second bacterial species compared to a reconstituted bacterial stock consisting essentially of the at least one of a second bacterial species. In some embodiments, when the cryopreserved bacterial culture is reconstituted, the reconstituted cryopreserved bacterial culture has at least 10,000× increased bacterial growth measured in colony forming units per mL (cfu/mL) of the at least one of a second bacterial species compared to a reconstituted bacterial stock consisting essentially of the at least one of a second bacterial species. In some embodiments, when the cryopreserved bacterial culture is reconstituted, the reconstituted cryopreserved bacterial culture has at least 100,000× increased bacterial growth measured in colony forming units per mL (cfu/mL) of the at least one of a second bacterial species compared to a reconstituted bacterial stock consisting essentially of the at least one of a second bacterial species. In some embodiments, when the cryopreserved bacterial culture is reconstituted, the reconstituted cryopreserved bacterial culture has at least 1,000,000× increased bacterial growth measured in colony forming units per mL (cfu/mL) of the at least one of a second bacterial species compared to a reconstituted bacterial stock consisting essentially of the at least one of a second bacterial species. In some embodiments, when the cryopreserved bacterial culture is reconstituted, the reconstituted cryopreserved bacterial culture has at least 10,000,000× increased bacterial growth measured in colony forming units per mL (cfu/mL) of the at least one of a second bacterial species compared to a reconstituted bacterial stock consisting essentially of the at least one of a second bacterial species.

### Bacterial Composition

In an aspect, a composition comprising a cryopreservation formulation is presented, comprising:
a mixture of bacterial species in a manmade cryopreservation medium, the mixture comprising
   a) a first bacterial species, wherein the first bacterial species is Acidaminococcus intestini or Acidaminococcus fermentans; and
   b) at least one of a second bacterial species,
   wherein the first bacterial species is present in the cryopreservation formulation in an amount sufficient to confer cryoprotection to the at least one of the second bacterial species upon reconstitution of the manmade cryopreservation formulation, and
   wherein the manmade cryopreservation formulation after reconstitution exhibits at least 10× increased bacterial proliferation of the at least one of the second bacterial species in a bacterial proliferation assay relative to bacterial proliferation of a manmade cryopreservation formulation after reconstitution comprising the at least one of the second bacterial species absent the first bacterial species.
      As used herein, a manmade cryopreservation medium refers to a synthetic medium that is suitable for cryopreserving cells (e.g., bacterial cells) and is made by the “hand of man”.

### Bacterial Proliferation Assays

Various assays are known in the art for determining bacterial cell viability and proliferative capacity. In an exemplary embodiment, the bacterial proliferation assay involves streaking/plating on a suitable substrate (e.g., agar plate comprising suitable bacterial growth media) and incubating the plate under conditions suited for growth of the bacteria in question. In a particular embodiment, the plate is incubated under anaerobic conditions. See, e.g., Examples presented herein.

In another exemplary embodiment, the bacterial proliferation assay involves cell sorting. Flow cytometry is used to the analyze viability, metabolic state, and antigenic markers of bacteria. Flow cytometry is routinely used to determine the number of viable bacteria in a sample. Live cells have intact membranes and are impermeable to dyes such as propidium iodide (PI), which only leaks into cells with compromised membranes. Thiazole orange (TO), for example, is a permeant dye and enters all cells, live and dead, to varying degrees. With gram-negative organisms, depletion of the lipopolysaccharide layer with EDTA facilitates TO uptake. Thus, a combination of these two dyes provides a rapid and reliable method for discriminating live and dead bacteria. If enumeration of the bacteria is important, BD Biosciences Liquid Counting Beads (BD Biosciences, San Jose, Calif.), a flow cytometry bead standard, can be used to accurately quantify the number of live, dead, and total bacteria in a sample.

An exemplary protocol for flow cytometry is as follows:

### Bacteria:

For cultured bacteria, dilute to an approximate concentration range of 5×10⁵ to 9×10⁶ bacteria/mL in staining buffer. To prepare killed bacteria, mix 0.5 mL of culture before dilution with 0.5 ml of SPOR-KLENZ™ (Steris Corporation, St. Louis, Mo., Catalog No. 6525-01) disinfectant for 5 minutes.

Staining:

1. Label 12×75-mm polystyrene tubes.
2. Vortex bacterial suspension or sample and dilute at least 1:10 in staining buffer.
3. Add 200 μL of bacterial suspension, diluted as above in staining buffer.
4. Add 5.0 μL of each dye solution to the tubes. The final staining concentrations are 420 nM for TO and 48 μM for PI.
5. Vortex and incubate for 5 minutes at room temperature.
6. Reverse pipet 50 μL of BD Liquid Counting Beads into the staining tube to determine the concentration of live, dead and total bacteria.
7. Analyze on, e.g., a BD FACS brand flow cytometer (BD FACSCalibur flow cytometer or equivalent).

Flow Cytometer Setup:

1. Use BD CaliBRITE™ 3 beads (BD Biosciences Catalog No. 349502) and the appropriate software, such as BD FACSComp™ or BD AutoCOMP™ software, for setting the photomultiplier tube (PMT) voltages and the fluorescent compensations, and for checking instrument sensitivity prior to use.

2. Initial instrument settings should be as follows:

### Threshold—SSC

FSC—E01, logarithmic amplification
SSC—375 V, logarithmic amplification
FL1—600 V, logarithmic amplification
FL3—800 V, logarithmic amplification
Compensation—none used

3. Actual settings can vary with the application and should be optimized as follows: Set threshold on side scatter (SSC), and adjust PMT voltages and threshold levels using an unstained sample of diluted bacteria. The bacterial population should be positioned so that it is entirely on scale on an FSC vs SSC plot (FIG. 1A). Individual FSC and SSC histograms should be checked to be sure that the bell-shaped populations are visible. If the entire population is not present, adjust PMT values to position the peak on the histograms and decrease the threshold until the entire population is visible. As the voltage is further increased, the background noise should become evident on the lower end of the histogram. The balance of PMT voltage and threshold should allow the entire peak to be observed with at least a portion of the valley between the bacteria and the noise. Actual peak shapes and resolution from noise will vary with bacterial morphology and sample matrix.

4. Set FL1 and FL3 PMT voltages to place the unstained population in the lower left quadrant of an FL1 vs FL3 plot.

Data Acquisition and Analysis:

1. Acquire prepared samples on a BD FACS brand flow cytometer using an SSC threshold. Acquire data with BD CellQuest™ Pro or BD LYSYS™ II software, in Acquisition-to-Analysis mode. Set up an FSC vs SSC plot with a live gate around the bacterial population R1. If BD Liquid Counting Beads are used, set a region R2 around the beads on the FSC vs SSC plot. Set another region R3 around the stained bacterial population in the FL2 vs SSC dot plot and an FL1 vs FL3 plot, gated on combined parameters FSC, SSC, and FL2 (FL1 vs FL3 gated on [R1 OR R2] AND R3) to display the stain results (FIG. 1C).

2. Acquire a total of 10,000 events.

3. In Analysis mode, draw rectilinear regions around the live, dead, and injured populations.

4. If BD Liquid Counting Beads used, determine the absolute count.

Controls:

Use an unstained bacterial sample to confirm that PMT voltages are set appropriately. Dilute, stain, and acquire an aliquot of culture media or sample matrix, diluted the same as a bacterial sample, to confirm that assay background is low. Use a mixture of live and killed bacteria to confirm that stained live, injured, and dead bacterial populations are sufficiently resolved.

In addition to PI, other vital stains may be used such as, without limitation, ethidium bromide, fluorescein diacetate, and acridine may be used in flow cytometry to determine the number of live/dead bacterial cells.

### EXAMPLES

### Example 1: Strain Survivability

A number of microbes derived from human feces exhibit sensitivity to freezing and lyophilization as evidenced by reduced viability and, in extreme cases, failure survive these processes. This presents a problem because it alters the microbial diversity of populations that can be generated from fecal samples, thereby rendering such microbial populations non-representative of the originating material. In circumstances wherein a species that has beneficial properties exhibits sensitivity to freezing and lyophilization, it limits and/or prohibits the ability to maintain stocks of the sensitive species, thereby requiring regular access to freshly isolated supplies of the sensitive species from primary sources.

To freeze, vials containing 1 mL aliquots of co-culture described herein were placed in a −80° C. freezer and frozen for at least 24 hours. To lyophilize, the lyophilizer used for the freeze drying process was the Labconco Freeze Dry System/Freezone 4.5, 7750000. To reconstitute the microbes, the following protocol was used:

(1) Reconstitution medium (1×PBS) was placed in an anaerobic chamber overnight to degas the medium. The entirety of the reconstitution protocol was conducted in the anaerobic chamber. A 1:1 ratio of co-culture volume to reconstitution medium is used to reconstitute co-cultures. If, for example, 1 mL of co-cultured volume was frozen and lyophilized, then 1 mL was used to reconstitute the culture.

(2) 1 mL of reconstitution medium was aliquoted into the vial comprising a frozen co-culture and left in the anaerobic chamber for 15 minutes. The vial was inverted every few minutes to ensure a thoroughly mixed culture.

(3) This culture was then plated to determine cfu/mL.

Even with the use of a cryo-protectant and lyo-protectant medium, sensitive strains such as those presented in Table 1 had reconstitution values at or under 10² cfu/mL. All eight strains (which come from the MET-1 defined community derived from human feces as described in US20140363397) also experienced batch to batch inconsistencies, sometimes not growing back from lyophilization even at a neat dilution.

TABLE 1
Reconstitution cfu/mL results for eight strains derived
from human feces (MET-1) following lyophilization:

| | Starting | Reconstitution cfu/mL | | |
|---|---|---|---|---|
| Strain | cfu/mL | Run 1 | Run 2 | Run 3 |
| 43 FAA (Roseburia hominis) | 1.7 × 10¹¹ | NG* | 2.3 × 10¹ | NG |
| F1 FAA (Eubacterium eligens) | 4.3 × 10¹² | NG | NG | 5.8 × 10¹ |
| 6 FM (Eubacterium rectale) | 1.8 × 10¹¹ | 1.3 × 10² | NG | 3.5 × 10¹ |
| 1 FAA (Eubacterium rectale) | 1.3 × 10¹⁰ | 1.5 × 10¹ | 3.0 × 10² | NG |
| 29 FAA (Eubacterium rectale) | 3.7 × 10¹⁰ | 3.2 × 10² | NG | NG |
| 18 FAA (Eubacterium rectale) | 4.0 × 10⁹ | NG | NG | NG |
| 30 FAA (Ruminococcus torques) | 2.1 × 10¹¹ | NG | 8.4 × 10² | 2.0 × 10² |
| 39 FAA (Roseburia faecis) | 1.5 × 10¹¹ | NG | NG | NG |

*NG—No growth of an undiluted aliquot of a bacterial culture on a fastidious anaerobe agar (FAA) plate

### Example 2: Co-Culture with Acidaminococcus intestini (14 LG)

Dilution series of overnight cultures of 6 FM, 43 FAA, F1 FAA, 1 FAA, 29 FAA, 18 FAA, 39 FAA and 30 FAA (from MET-1) were plated on FAA to determine the starting cfu/mL (see, e.g., Table 2). All eight strains were then individually co-cultured with an overnight culture of Acidaminococcus intestini 14 LG (OD₆₀₀=0.782) in equal parts (10 mL:10 mL) for 2 hours. Cultures were then centrifuged and resuspended in 5% riboflavin/cysteine/inulin at a concentration of 10% solids. Samples were aliquoted into 1 mL volumes and frozen at −80° C. overnight. Samples were then lyophilized, reconstituted and plated to determine the recovery cfu/mL. Mixed cultures were observed for all eight strains (this was expected and suggests the presence of both 14 LG and the strain of interest). A difference in colony morphology was observed between 14 LG and each strain of interest and their individual identities were confirmed by Sanger sequencing. Colonies from the strain of interest were enumerated and can be used as an approximate recovery cfu/mL (see, e.g., Table 2).

TABLE 2
Reconstitution cfu/mL results for eight strains following co-culture
with 14 LG. Results are the average values from triplicate runs:

| | | | Reconstitution cfu/mL |
|---|---|---|---|
| | Starting | Starting | (adjusted for |
| Strain | OD₆₀₀ | cfu/mL | concentration spin steps) |
| 1 FAA (Eubacterium | 0.156 | 1.8 × 10¹⁰ | 2.0 × 10⁶ |
| rectale) | | | |
| 29 FAA (Eubacterium | 0.234 | 2.4 × 10¹¹ | 6.0 × 10⁶ |
| rectale) | | | |
| 18 FAA (Eubacterium | 0.225 | 5.0 × 10¹¹ | 1.0 × 10⁶ |
| rectale) | | | |
| 30 FAA | 0.529 | 1.4 × 10¹¹ | 5.0 × 10⁸ |
| (Ruminococcus | | | |
| torques) | | | |
| 39 FAA (Rosehuria | 1.35 | 1.2 × 10¹¹ | 2.0 × 10⁶ |
| faecis) | | | |
| F1 FAA (Eubacterium | 0.774 | 6.0 × 10¹² | 1.1 × 10⁷ |
| eligens) | | | |
| 43 FAA (Roseburia | 0.284 | 2.0 × 10¹¹ | 3.0 × 10⁷ |
| hominis) | | | |
| 6 FM (Eubacterium | 0.148 | 1.6 × 10¹¹ | 8.5 × 10⁶ |
| rectale) | | | |

Upon reconstitution, all eight strains not only survived the freezing and lyophilization conditions but did so with predictable robustness (e.g., but not limited to, at least 10%, 20%, 30%, 40%, 50%, 60%, 70%, 80%, or 90% bacterial species survived and grew on a FAA plate).

Co-culture with 14 LG was also conducted with a cohort of strains isolated from a different fecal donor (“NB2”—a healthy, 28 year old male individual of average body mass index (BMI), who had previously undergone health screening as part of a program to allow him to become a FMT donor in Canada). Of the 39 strains, 21 did not grow back at a 10,000× or (4-fold serial) dilution of at least 10⁻⁴ from freezing and lyophilization using traditional cryo- and lyo-protectant methods. These strains were co-cultured individually with an overnight culture of 14 LG in equal parts (10 mL:10 mL) for 2 hours. Cultures were then centrifuged and resuspended in 5% riboflavin/cysteine/inulin at a concentration of 10% solids. Samples were aliquoted into 1 mL volumes and frozen at −80 degrees C. overnight. Samples were then lyophilized, reconstituted and an aliquot was plated undiluted on a FAA plate to determine the recovery cfu/mL. Mixed cultures were observed for all 21 strains (this was expected and suggests the presence of both 14 LG and the strain of interest). A difference in colony morphology was observed between 14 LG and each strain of interest and their individual identities were confirmed by Sanger sequencing. The strain of interest colonies were counted and can be used as an approximate recovery cfu/mL (see, e.g., Table 3).

TABLE 3
Reconstitution cfu/mL results for 21 strains from
donor NB2 following co-culture with 14 LG. Results
are the average values from triplicate runs:

| | | Reconstitution cfu/mL |
|---|---|---|
| | | (adjusted for concentration |
| Strain | OD₆₀₀ | spin steps) |
| A-2 FAA (Coprococcus comes) | 1.07 | 5.6 × 10⁹ |
| B-15 DCM (Dorea | 0.535 | 8.0 × 10⁷ |
| formicigenerans) | | |
| B-13 CNA (Eubacterium | 0.397 | 2.0 × 10⁸ |
| contortum) | | |
| B-17 NB (Ruminococcus | 0.317 | 1.3 × 10⁸ |
| lactaris) | | |
| A-17 FMU (Eubacterium | 0.189 | 9.2 × 10⁵ |
| rectale) | | |
| B-19 DCM (Faecalibacterium | 0.181 | 3.4 × 10⁵ |
| prausnitzii) | | |
| B-6 CNA (Eubacterium eligens) | 0.885 | 3.5 × 10¹¹ |
| A-14 FMU (Ruminococcus | 0.207 | 2.2 × 10⁸ |
| torques) | | |
| B-10 FAA (Roseburia | 0.236 | 8.6 × 10⁷ |
| intestinalis) | | |
| B-9 DCM (Anaerostipes | 1.30 | 1.0 × 10⁵ |
| hadrus) | | |
| B-9 FAA (Blautia luti) | 1.009 | 3.4 × 10⁷ |
| A-9 NA (Ruminococcus obeum) | 1.058 | 7.5 × 10⁶ |
| A-5 TSAB (Blautia stercoris) | 0.361 | 2.0 × 10⁷ |
| A-3 NA (Dorea longicatena) | 0.887 | 3.1 × 10⁷ |
| B-10 NB (Clostridium | 0.279 | 1.3 × 10⁸ |
| spiroforme) | | |
| B-10 MRS (Eubacterium | 1.018 | 2.5 × 10⁸ |
| desmolans) | | |
| A-14 DS (Clostridium | 0.276 | 1.2 × 10⁸ |
| aerotolerans) | | |
| B-20 GAM (Clostridium | 0.225 | 2.1 × 10⁸ |
| lactatifermen tans) | | |
| B-13 BHI (Eubacterium hallii) | 1.30 | 3.2 × 10⁴ |
| B-20 DS (Clostridium | 0.190 | 5.0 × 10⁵ |
| hylemonae) | | |
| B-26 FMU (Roseburict | 0.203 | 1.7 × 10⁵ |
| inulinivorans) | | |

Upon reconstitution, all 21 strains from donor NB2 not only survived the freezing and lyophilization conditions, but also did so with predictable robustness (e.g., but not limited to, at least 10%, 20%, 30%, 40%, 50%, 60%, 70%, 80%, or 90% bacterial species survived and grew on a FAA plate).

### Example 3: Co-Culture with Filter-Sterilized Supernatants of Acidaminococcus intestini 14 LG

Overnight cultures of 43 FAA, F1 FAA and 6 FM were plated using an undiluted aliquot on a FAA plate to determine the starting cfu/mL (see, e.g., Table 4). 43 FAA, F1 FAA and 6 FM were then individually co-cultured with the filter-sterilized (0.22 μm filter) supernatant of an overnight culture of 14 LG (OD₆₀₀=0.763) in equal parts (10 mL:10 mL) for 2 hours. Cultures were then centrifuged at 4000 rpm and resuspended in 5% riboflavin/cysteine/inulin at a concentration of 10% solids. Samples were aliquoted into 1 mL volumes and frozen at −80 degrees C. overnight. Samples were then lyophilized, reconstituted and plated to determine the recovery cfu/mL. On all reconstitution plates, including the neat plates, for all strains there was no growth observed (see, e.g., Table 4). This suggests that the protective nature of 14 LG is likely the result of an interaction between live microbes and not their secreted products.

TABLE 4
Reconstitution cfu/mL results for three strains following
co-culture with filter-sterilized 14 LG supernatant.
Results are the average values from triplicate runs:

| | Starting | Starting | Reconstitution |
|---|---|---|---|
| Strain | OD₆₀₀ | cfu/mL | cfu/mL |
| 43 FAA (Roseburia | 0.322 | 2.2 × 10¹¹ | No Growth* |
| hominis) | | | |
| F1 FAA (Eubacterium | 1.035 | 2.4 × 10¹² | No Growth |
| eligens) | | | |
| 6 FM (Eubacterium | 0.153 | 1.9 × 10¹¹ | No Growth |
| rectale) | | | |

*No growth of an undiluted aliquot of a bacterial culture on a fastidious anaerobe agar (FAA) plate

### Example 4: Co-Culture with Other Strains of Acidaminococcus intestini (GAM 7, CC1/6 D9)

Other strains of Acidaminococcus intestini isolated from the fecal samples of different donors were also tested. GAM 7 is a strain of A. intestini that was isolated from the fecal sample of an obese individual and CC1/6 D9 is a strain of A. intestini that was isolated from the intestinal biopsy of an individual with colorectal cancer. Overnight cultures of 1 FAA and 39 FAA were individually co-cultured with overnight cultures of either GAM 7 (OD₆₀₀=0.776) or CC1/6 D9 (OD₆₀₀=1.216) in equal parts (10 mL:10 mL) for 2 hours. Cultures were then centrifuged and resuspended in 5% riboflavin/cysteine/inulin at a concentration of 10% solids. Samples were aliquoted into 1 mL volumes and frozen at −80 degrees C. overnight. Samples were then lyophilized, reconstituted and were plated using an undiluted aliquot on a FAA plate to determine the recovery cfu/mL. Colonies were counted, picked and delivered for Sanger sequencing to determine closest species identity. Co-culture with either GAM 7 or CC1/6 D9 resulted in relatively robust and consistent reconstitution values for both 1 FAA and 39 FAA, equating or surpassing those values observed when co-cultured with 14 LG (see, e.g., Tables 5 and 6). These results suggest that the protective nature of co-culturing with A. intestini prior to freezing and lyophilization is an ability associated with the species rather than the specific strain.

TABLE 5
Reconstitution cfu/mL results for two strains following co-culture
with GAM 7. Results are the average values from triplicate runs:

| Strain | Starting OD₆₀₀ | Reconstitution cfu/mL |
|---|---|---|
| 1 FAA (Eubacterium | 0.411 | 6.25 × 10⁶ |
| rectale) | | |
| 39 FAA (Rosehuria | 1.37 | 7.1 × 10⁵ |
| faecis) | | |

TABLE 6
Reconstitution cfu/mL results for two strains following co-culture
with CC1/6 D9. Results are the average values from triplicate runs:

| Strain | Starting OD₆₀₀ | Reconstitution cfu/mL |
|---|---|---|
| 1 FAA (Eubacterium | 0.411 | 5.3 × 10⁷ |
| rectale) | | |
| 39 FAA (Roseburia | 1.37 | 4.1 × 10⁷ |
| faecis) | | |

### Example 5: Co-Culture with Strains Other than Acidaminococcus intestini (25 MRS, 5 MM and 12 FMU)

Alternative microbes to A. intestini were selected for co-culture to determine if protection during freezing and lyophilization is a trait specific to A. intestini or is simply the by-product of co-culture with other microbes. Lactobacillus casei (25 MRS) and Bacteroides ovatus (5 MM) were selected from our MET-1 list of microbes and Phascolarctobacterium succinatutens (12 FMU) was selected from our NB2 list of microbes to serve as alternatives to A. intestini for co-culture. Overnight cultures of 1 FAA and 39 FAA (from MET-1) were individually co-cultured with overnight cultures of either 25 MRS (OD₆₀₀=1.3) or 5 MM (OD₆₀₀=1.3) in equal parts (10 mL:10 mL) for 2 hours. Additionally, 14 FMU, 9 NA, 17 FMU and 5 TSAB (from NB2) were individually co-cultured with overnight cultures of 12 FMU (OD₆₀₀=0.166) in equal parts (10 mL:10 mL) for 2 hours. Cultures were then centrifuged and resuspended in 5% riboflavin/cysteine/inulin at a concentration of 10% solids. Samples were aliquoted into 1 mL volumes and frozen at −80 degrees C. overnight. Samples were then lyophilized, reconstituted and plated using an undiluted aliquot on a FAA plate to determine the recovery cfu/mL. Colonies were counted, picked and delivered for Sanger sequencing to determine closest species match/identity. In co-culturing of 39 FAA and 1 FAA with either 5 MM or 25 MRS there was no growth of either 39 FAA or 1 FAA observed upon reconstitution (see, e.g., Tables 7 and 8). Likewise co-culture of 12 FMU with 14 FMU, 9 NA, 17 FMU or 5 TSAB resulted in no observed growth of 14 FMU, 9 NA, 17 FMU or 5 TSAB upon reconstitution (see, e.g., Table 9). These results suggest that the protective nature of co-culturing with A. intestini prior to freezing and lyophilization is an ability associated with A. intestini and not simply the result of the co-culture of any two microbes.

TABLE 7
Reconstitution cfu/mL results for two strains following co-culture
with 25 MRS. Results are the average values from triplicate runs.

| Strain | Starting OD₆₀₀ | Reconstitution cfu/mL |
|---|---|---|
| 1 FAA (Eubacterium | 0.411 | No Growth* |
| rectale) | | |
| 39 FAA (Roseburia | 1.37 | No Growth |
| faecis) | | |

*No growth of an undiluted aliquot of a bacterial culture on a fastidious anaerobe agar (FAA) plate

TABLE 8
Reconstitution cfu/mL results for two strains following co-culture
with 5 MM. Results are the average values from triplicate runs.

| Strain | Starting OD₆₀₀ | Reconstitution cfu/mL |
|---|---|---|
| 1 FAA (Eubacterium | 0.170 | No Growth* |
| rectale) | | |
| 39 FAA (Roseburia | 1.33 | No Growth |
| faecis) | | |

*No growth of an undiluted aliquot of a bacterial culture on a fastidious anaerobe agar (FAA) plate

TABLE 9
Reconstitution cfu/mL results for two strains following co-culture
with 12 FMU. Results are the average values from triplicate runs.

| Strain | Starting OD₆₀₀ | Reconstitution cfu/mL |
|---|---|---|
| 14 FMU | 0.207 | No Growth* |
| (Ruminococcus | | |
| torques) | | |
| 9 NA (Ruminococcus | 1.058 | No Growth |
| obeum) | | |
| 17 FMU (Eubacterium | 0.089 | No Growth |
| rectale) | | |
| 5 TSAB (Blautia | 0.361 | No Growth |
| stercoris) | | |

*No growth of an undiluted aliquot of a bacterial culture on a fastidious anaerobe agar (FAA) plate

### Example 6: Co-Culture with 14 LG without the Use of Cryo/Lyo-Protectants

Co-culturing was done without the use of a cryo-protectant or lyo-protectant medium to determine if co-culturing with 14 LG is enough to promote the survival of strains that are sensitive to freezing/lyophilization. Overnight cultures of 1 FAA and 39 FAA were individually co-cultured with an overnight culture of 14 LG (OD₆₀₀=0.633) in equal parts (10 mL:10 mL) for 2 hours. Cultures were then centrifuged and resuspended in ddH₂O at a concentration of 10% solids. Samples were aliquoted into 1 mL volumes and frozen at −80 degrees C. overnight. Samples were then lyophilized, reconstituted and plated using an undiluted aliquot on a FAA plate to determine the recovery cfu/mL. Colonies were counted, picked and delivered for Sanger sequencing to determine identity using the processes described herein. Co-culture of 14 LG without the use of any cryo/lyo-protectant medium resulted in the consistent survival of both 1 FAA and 39 FAA (see, e.g., Table 10). However, when co-culture with 14 LG and a cryo/lyo-protectant medium are used in combination, both 1 FAA and 39 FAA had higher reconstitution values than with co-culture alone (see, e.g., Tables 2 and 10). These results demonstrate that co-culture with 14 LG is enough to improve the survivability of sensitive microbes during freezing and lyophilization, although increased growth is observed upon further supplementation with a cryo/lyo-protectant medium (e.g., but not limited to, 100×, 1,000×, 10,000×, 100,000× improved survivability of sensitive microbes when using cryo/lyo-protectant medium).

TABLE 10
Reconstitution cfu/mL results for two strains following co-culture
with 14 LG without using any cryo-protectant or lyo-protectant.
Results are the average values from triplicate runs:

| Strain | Starting OD₆₀₀ | Reconstitution cfu/mL |
|---|---|---|
| 1 FAA (Eubacterium | 0.411 | 7.7 × 10⁵ |
| rectale) | | |
| 39 FAA (Roseburia | 1.37 | 1.2 × 10² |
| faecis) | | |

### Example 7: Co-Culture with Killed 14 LG

Co-culturing with killed 14 LG was conducted to determine if its protective properties are the result of an interaction that takes place between live microbes. An overnight culture of 14 LG (OD₆₀₀=0.676) was boiled for 20 minutes to destroy all live cells. This culture was then plated using an undiluted aliquot on a FAA plate to ensure that no growth was observed. Overnight cultures of 1 FAA and 39 FAA were individually co-cultured with boiled 14 LG in equal parts (10 mL:10 mL) for 2 hours. Cultures were then centrifuged and resuspended in 5% riboflavin/cysteine/inulin at a concentration of 10% solids. Samples were aliquoted into 1 mL volumes and frozen at −80 degrees C. overnight. Samples were then lyophilized, reconstituted and plated to determine the recovery cfu/mL. No growth was observed at any dilution, including on neat plates, (e.g., undiluted aliquot of a bacterial culture on fastidious anaerobe agar (FAA) plates) (see, e.g., Table 11). This observation indicates that the protective nature of 14 LG co-culturing is the result of an interaction that takes place between live microbes. Alternatively, the boiling procedure altered or destroyed some physical feature of 14LG cells that plays a role in the protective property.

TABLE 11
Reconstitution cfu/mL results for two strains following co-culture with
killed 14 LG. Results are the average values from triplicate runs.

| Strain | Starting OD₆₀₀ | Reconstitution cfu/mL |
|---|---|---|
| 1 FAA (Eubacterium | 0.170 | No Growth* |
| rectale) | | |
| 100X39 FAA | 1.33 | No Growth |
| (Roseburia faecis) | | |

*No growth of an undiluted aliquot of a bacterial culture on a fastidious anaerobe agar (FAA) plate

### Example 8: Alternative Timing and Concentrations for 14 LG Co-Culturing

In all of the experiments conducted, co-culturing of bacterial isolates with 14 LG took place in equal parts for 2 hours. However, co-culturing was also tested at different dilutions and after different durations. Dilutions of 1:20 (14 LG:Strain X) and 1:10 (14 LG:Strain X) were tested for each of the eight sensitive microbes at 0, 30 minute and 1 hour time points, respectively. Overnight cultures of 6 FM, 43 FAA, F1 FAA, 1 FAA, 29 FAA, 18 FAA, 39 FAA and 30 FAA were grown, co-cultured with 14 LG at the appropriate dilution and for the appropriate duration, and then processed as previously described. Samples were reconstituted and plated to determine the recovery cfu/mL. Although the results differ for each strain, there is a trend indicating that reconstitution values improve as the duration of the co-culture and the concentration of 14 LG increase (see, e.g., Table 12). This suggests that the mechanism employed by 14 LG to protect sensitive microbes during freezing and lyophilization requires time in order to function optimally.

TABLE 12
Reconstitution cfu/mL results for all 8 strains following co-culture
with various concentrations of 14 LG for different durations:

| | | 1:20 (14 LG:Strain X) | | | 1:10 (14 LG:Strain X) | | |
|---|---|---|---|---|---|---|---|
| | Starting | 0 | 30 | 1 | 0 | 30 | 1 |
| Strain | OD₆₀₀ | min. | min. | hr | min. | min. | hr |
| 43 FAA (Rosebuna | 0.413 | NG | 2.6 × 10³ | NG | 4.5 × 10⁷ | 1.1 × 10⁸ | 1.2 × 10⁸ |
| hominis) | | | cfu/mL | | cfu/mL | cfu/mL | cfu/mL |
| F1 FAA (Eubacterium | 1.11 | NG | 5.7 × 10³ | 8.0 × 10² | 6.0 × 10⁶ | 4.9 × 10⁷ | 1.2 × 10^{s} |
| eligens) | | | cfu/mL | cfu/mL | cfu/mL | cfu/mL | cfu/mL |
| 6 FM (Eubacterium | 0.481 | 8.0 × 10² | 3.7 × 10³ | 8.0 × 10⁶ | 2.5 × 10³ | 1.7 × 10⁷ | 1.0 × 10⁷ |
| rectale) | | cfu/mL | cfu/mL | cfu/mL | cfu/mL | cfu/mL | cfu/mL |
| 1 FAA (Eubacterium | 0.322 | NG | 1.3 × 10² | NG | NG | NG | 1.04 × 10³ |
| rectale) | | | cfu/mL | | | | cfu/mL |
| 29 FAA (Eubacterium | 0.466 | NG | NG | 5.0 × 10¹ | NG | 6.0 × 10³ | 1.5 × 10³ |
| rectale) | | | | cfu/mL | | cfu/mL | cfu/mL |
| 18 FAA (Eubacterium | 0.318 | NG | NG | NG | NG | 5.0 × 10² | 1.5 × 10³ |
| rectale) | | | | | | cfu/mL | cfu/mL |
| 30 FAA (Ruminococcus | 0.525 | 2.6 × 10² | 6.0 × 10⁶ | 1.5 × 10⁶ | 2.0 × 10⁶ | 1.5 × 10⁷ | 3.1 × 10⁷ |
| torques) | | cfu/mL | cfu/mL | cfu/mL | cfu/mL | cfu/mL | cfu/mL |
| 39 FAA (Rosehuria | 1.34 | NG | NG | NG | NG | NG | 2.0 × 10² |
| faecis) | | | | | | | cfu/mL |

*NG—No growth of an undiluted aliquot of a bacterial culture on a fastidious anaerobe agar (FAA) plate

### Example 9: Co-Culture with Closely Related Acidaminococcus fermentans (DSM 20731)

Acidaminococcus fermentans was selected for co-culture testing to determine if protection conferred during freezing and lyophilization is a trait also shared by Acidaminococcus intestini's closest relative on the All Species Living Tree (16S rRNA-based phylogenetic tree). B-6 CNA (a Eubacterium eligens derived from NB2), B-10 FAA (a Roseburia intestinalis derived from NB2), DSM 20731 (Acidaminococcus fermentans from the DSMZ strain bank), 14 LG (Acidaminococcus intestini isolated from MET-1) and DSM 21505 (Acidaminococcus intestini from the DSMZ strain bank) were used in this experiment. Overnight cultures of B-6 CNA (OD₆₀₀=0.8) and B-10 FAA (OD₆₀₀=0.232) were individually co-cultured with overnight cultures of either DSM 20731 (OD₆₀₀=0.685), 14 LG (OD₆₀₀=0.777) or DSM 21505 (OD₆₀₀=0.762) in equal parts (10 mL:10 mL) for 2 hours. Cultures were then spun down and re-suspended in 5% riboflavin/cysteine/inulin at a concentration of 10% solids. Samples were aliquoted into 1 mL volumes and frozen at −80° C. overnight. Samples were then lyophilized, reconstituted and plated to determine the recovery cfu/mL. Colonies were counted, picked and delivered for Sanger sequencing to determine identity. Co-culture with A. fermentans, although not as robust as A. intestini for B-6 CNA, still allowed for consistent recovery of both B-6 CNA and B-10 FAA in comparison to freezing and lyophilization without any co-culturing (Tables 13 and 14). These results suggest that although A. fermentans may not offer as robust protection for some microbes as A. intestini it does still allow for consistent recovery of microbes that would otherwise not survive freezing and lyophilization.

TABLE 13
Reconstitution cfu/mL results for B-6 CNA following
co-culture with DSM 20731, 14 LG or DSM 21505. Results
are the average values from triplicate runs.

| Strain | Reconstitution cfu/mL |
|---|---|
| DSM 20731 (A. fermentans) | 4.5 × 10³ |
| 14 LG (A. intestini) | 2.9 × 10⁵ |
| DSM 21505 (A. intestini) | 9.8 × 10⁶ |
| No Co-Culture Partner | No Growth* |

*No growth on a neat plate.

TABLE 14
Reconstitution cfu/mL results for B-10 FAA following
co-culture with DSM 20731, 14 LG or DSM 21505. Results
are the average values from triplicate runs.

| Strain | Reconstitution cfu/mL |
|---|---|
| DSM 20731 (A. fermentans) | 5.8 × 10⁵ |
| 14 LG (A. intestini) | 5.0 × 10⁵ |
| DSM 21505 (A. intestini) | 1.8 × 10⁶ |
| No Co-culture Partner | No Growth* |

*No growth on a neat plate.

### Example 10: Is Cell-to-Cell Contact Required for A. Intestini Cryo/Lyo Protection?

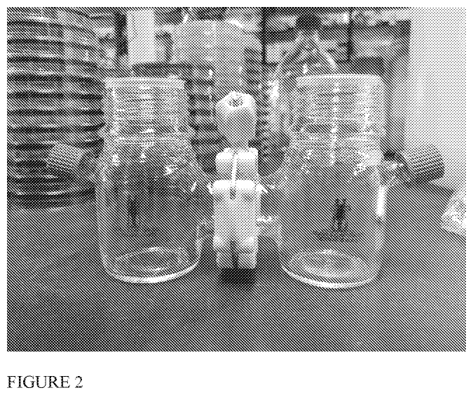

It is unknown whether cell-to-cell contact is required during the co-culture process with A. intestini to confer cryo/lyo protection. To investigate this issue, a co-culturing double flask apparatus was used (see FIG. 2).

B-6 CNA (Eubacterium eligens from NB2) was tested in three distinct ways. First, an overnight culture of B-6 CNA was spun down and re-suspended in 5% riboflavin/cysteine/inulin at a concentration of 10% solids. Second, an overnight culture of B-6 CNA was co-cultured with an overnight culture of 14 LG (A. intestini from MET-1) in equal parts (10 mL:10 mL) for 2 hours. Cultures were then spun down and re-suspended in ddH₂O at a concentration of 10% solids. Third, 100 mL of an overnight culture of B-6 CNA was co-cultured with 100 mL of an overnight culture of 14 LG in the co-culture double flask apparatus (i.e., B-6 CNA was in one side/bottle and 14 LG was in the other) for 2 hours. Samples from all three different treatment groups were then aliquoted into 1 mL volumes and frozen at −80° C. overnight. Samples were then lyophilized, reconstituted and plated to determine the recovery cfu/mL. B-6 CNA was only recovered when it was co-cultured in direct contact with 14 LG (Table 15). These findings suggest that the protective nature of A. intestini co-culture might be a result of direct cell-to-cell contact between A. intestini and its co-culture companion strain. These results also add evidence to those found in Example 7 above that describe the ineffectiveness of co-culturing with filter sterile A. intestini supernatant.

TABLE 15
Reconstitution cfu/mL results for B-6 CNA following
no co-culture and co-culture with 14 LG, either in
direct contact or through a double flask apparatus.
Results are the average values from triplicate runs.

| Strain | Reconstitution cfu/mL |
|---|---|
| 14 LG | No Growth* |
| (A. intestini), through a | |
| double flask apparatus | |
| 14 LG | 3.5 × 10⁶ |
| (A. intestini), direct contact | |
| No Co-Culture Partner | No Growth |

*No growth on a neat plate.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

While a number of embodiments of the present invention have been described, it is understood that these embodiments are illustrative only, and not restrictive.

1. A method for improving bacterial viability following cryopreservation, comprising:
a) combining a first bacterial species, wherein the first bacterial species is an Acidaminococcus species or a member of the Acidaminococcaceae family, with at least one of a second bacterial species to produce a bacterial mixture, wherein the first bacterial species is present in the bacterial mixture in an amount sufficient to confer cryoprotection to the at least one of the second bacterial species and wherein the member of the Acidaminococcaceae species is Succinispira mobilis;
b) culturing the bacterial mixture to produce a cultured bacterial mixture, wherein the culturing is for a period of time sufficient to confer the cryoprotection to the at least one of the second bacterial species in the cultured bacterial mixture; and
c) cryopreserving the cultured bacterial mixture to produce a cryopreserved bacterial culture; wherein the cryopreserved bacterial culture after reconstitution exhibits at least 10× increased bacterial proliferation of the at least one of the second bacterial species in a bacterial proliferation assay relative to bacterial proliferation of a cryopreserved bacterial culture after reconstitution comprising the at least one of the second bacterial species absent the first bacterial species.

2. The method of claim 1, wherein the Acidaminococcus species is Acidaminococcus intestini or Acidaminococcus fermentans.

3. The method of claim 1, wherein the amount of the first bacterial species sufficient to confer cryoprotection to the at least one of the second bacterial species in the bacterial mixture is between 10% and 50% of a total amount of bacteria in the bacterial mixture.

4. The method of claim 1, wherein the bacterial proliferation assay is a bacterial plating assay.

5-8. (canceled)

9. The method of claim 1, wherein the at least one of the second bacterial species is at least one of Faecalibacterium prausnitzii, Coprococcus comes, Dorea formicigenerans, Eubacterium contortum, Ruminococcus lactaris, Eubacterium rectale, Eubacterium eligens, Ruminococcus torques, Roseburia intestinalis, Anaerostipes hadrus, Blautia luti, Ruminococcus obeum, Blautia stercoris, Dorea longicatena, Clostridium spiroforme, Eubacterium desmolans, Clostridium aerotolerans, Clostridium lactatifermentans, Eubacterium hallii, Clostridium hylemonae, Roseburia inulinivorans, Roseburia hominis, and Roseburia faecis.

10. The method of claim 1, wherein the cryopreserving comprises freezing and lyophilization.

11. (canceled)

12. The method of claim 1, wherein the cryopreserved bacterial culture comprises a lyophilization-protectant medium.

13. The method of claim 12, wherein the lyophilization-protectant medium comprises at least one of sucrose, Ficoll 70, and polyvinylpyrrolidone.

14. The method of claim 1, wherein the cryopreserved bacterial culture comprises at least one of riboflavin, cysteine, and inulin.

15. The method of claim 1, wherein the cryopreserved bacterial culture comprises a cryo-protectant medium.

16-37. (canceled)

38. The method of claim 1, wherein a ratio of the first bacterial species to the at least one of the second bacterial species in the bacterial mixture is at least 1:10.

39-40. (canceled)

41. A composition comprising a cryopreservation formulation, comprising:
a mixture of bacterial species in a manmade cryopreservation medium, the mixture comprising
a) a first bacterial species, wherein the first bacterial species is Acidaminococcus intestini or Acidaminococcus fermentans; and
b) at least one of a second bacterial species,
wherein the first bacterial species is present in the cryopreservation formulation in an amount sufficient to confer cryoprotection to the at least one of the second bacterial species upon reconstitution of the manmade cryopreservation formulation, and
wherein the manmade cryopreservation formulation after reconstitution exhibits at least 10× increased bacterial proliferation of the at least one of the second bacterial species in a bacterial proliferation assay relative to bacterial proliferation of a manmade cryopreservation formulation after reconstitution comprising the at least one of the second bacterial species absent the first bacterial species.

42. The composition of claim 41, wherein the amount of the first bacterial species sufficient to confer cryoprotection to the at least one of the second bacterial species in the bacterial mixture is between 10% and 50% of a total amount of bacteria in the manmade cryopreservation formulation.

43. The composition of claim 41, wherein the bacterial proliferation assay is a bacterial plating assay.

44-47. (canceled)

48. The composition of claim 41, wherein the at least one of the second bacterial species is at least one of Faecalibacterium prausnitzii, Coprococcus comes, Dorea formicigenerans, Eubacterium contortum, Ruminococcus lactaris, Eubacterium rectale, Eubacterium eligens, Ruminococcus torques, Roseburia intestinalis, Anaerostipes hadrus, Blautia luti, Ruminococcus obeum, Blautia stercoris, Dorea longicatena, Clostridium spiroforme, Eubacterium desmolans, Clostridium aerotolerans, Clostridium lactatifermentans, Eubacterium hallii, Clostridium hylemonae, Roseburia inulinivorans, Roseburia hominis, and Roseburia faecis.

49-57. (canceled)

58. The composition of claim 41, further comprising pharmaceutically acceptable excipient.

59. A pharmaceutical composition comprising a cryopreservation formulation, comprising:
a mixture of bacterial species in a manmade cryopreservation medium, the mixture comprising
a) a first bacterial species, wherein the first bacterial species is Acidaminococcus intestini or Acidaminococcus fermentans; and
b) at least one of a second bacterial species, wherein the at least one of the second bacterial species is present in a therapeutically effective amount, and
wherein the first bacterial species is present in the cryopreservation formulation in an amount sufficient to confer cryoprotection to the at least one of the second bacterial species upon reconstitution of the manmade cryopreservation formulation, and
wherein the manmade cryopreservation formulation after reconstitution exhibits at least 10× increased bacterial proliferation of the at least one of the second bacterial species in a bacterial proliferation assay relative to bacterial proliferation of a manmade cryopreservation formulation after reconstitution comprising the at least one of the second bacterial species absent the first bacterial species; and
a pharmaceutically acceptable excipient.

60. A method for ameliorating symptoms of a gastrointestinal disease in a subject afflicted with the gastrointestinal disease, the method comprising administering the pharmaceutical composition of claim 59 to the subject.

61. The method of claim 60, wherein the gastrointestinal disease comprises at least one of dysbiosis of a gastrointestinal tract, a Clostridium difficile (Clostridioides difficile) infection, and inflammatory bowel disease, irritable bowel syndrome, and diverticular disease.

62. The method of claim 61, wherein the inflammatory bowel disease is at least one of Crohn's disease and ulcerative colitis.
